SEQUENCE LISTING (17 sequences)

SEQ ID NO: 1 - protein, 20 aa, Artificial Sequence
  Description of Artificial Sequence Synthetic peptide
RRANAALKAGELYKSILYGC

SEQ ID NO: 2 - protein, 11 aa, Artificial Sequence
  Description of Artificial Sequence Synthetic peptide
GELYKSILYGC

SEQ ID NO: 3 - protein, 11 aa, Artificial Sequence
  Description of Artificial Sequence Synthetic peptide
RLDGNEIKRGC

SEQ ID NO: 4 - protein, 15 aa, Artificial Sequence
  Description of Artificial Sequence Synthetic peptide
AHEEISTTNEGVMGC

SEQ ID NO: 5 - protein, 30 aa, Artificial Sequence
  Description of Artificial Sequence Synthetic polypeptide
NGVFKYRPRYFLYKHAYFYPPLKRFPVQGC

SEQ ID NO: 6 - protein, 10 aa, Artificial Sequence
  Description of Artificial Sequence Synthetic peptide
CQDSETRTFY

SEQ ID NO: 7 - protein, 9 aa, Artificial Sequence
  Description of Artificial Sequence Synthetic peptide
TKKTLRTGC

SEQ ID NO: 8 - protein, 30 aa, Artificial Sequence
  Description of Artificial Sequence Synthetic polypeptide
GLRSKSKKFRRPDIQYPDATDEDITSHMGC

SEQ ID NO: 9 - protein, 9 aa, Artificial Sequence
  Description of Artificial Sequence Synthetic peptide
SQNPVQPGC

SEQ ID NO: 10 - protein, 13 aa, Artificial Sequence
  Description of Artificial Sequence Synthetic peptide
SYIRIADTNITGC

SEQ ID NO: 11 - protein, 11 aa, Artificial Sequence
  Description of Artificial Sequence Synthetic peptide
SYIRIADTNIT

SEQ ID NO: 12 - protein, 8 aa, Artificial Sequence
  Description of Artificial Sequence Synthetic peptide
KELNLVYT

SEQ ID NO: 13 - protein, 10 aa, Artificial Sequence
  Description of Artificial Sequence Synthetic peptide
KELNLVYTGC

SEQ ID NO: 14 - protein, 12 aa, Artificial Sequence
  Description of Artificial Sequence Synthetic peptide
GSITTIDVPWNV

SEQ ID NO: 15 - protein, 14 aa, Artificial Sequence
  Description of Artificial Sequence Synthetic peptide
GSITTIDVPWNVGC

SEQ ID NO: 16 - protein, 21 aa, Artificial Sequence
  Description of Artificial Sequence Synthetic peptide
GRRANAALKAGELYKSILYGC

SEQ ID NO: 17 - protein, 14 aa, Artificial Sequence
  Description of Artificial Sequence Synthetic peptide
GSYIRIADTNITGC

(19) United States
(12) Patent Application Publication
(10) Pub. No.: US 2012/0100106 A1
(43) Pub. Date: Apr. 26, 2012

(54) COLLAGEN-BINDING SYNTHETIC PEPTIDOGLYCANS FOR WOUND HEALING

(75) Inventors: Alyssa Panitch, West Lafayette, IN (US); John E. Paderi, Lafayette, IN (US)

(73) Assignee: PURDUE RESEARCH FOUNDATION, West Lafayette, IN (US)

(21) Appl. No.: 13/318,710

(22) PCT Filed: May 4, 2010
(86) PCT No.: PCT/US10/33543

§ 371 Date: Nov. 3, 2011

Related U.S. Application Data
(60) Provisional application No. 61/175,200, filed on May 4, 2009.

(51) Int. Cl. A61K 38/16; A61P 17/02

(52) U.S. Cl. 424/93.1; 514/18.6

(57) ABSTRACT

Methods and compositions for promoting wound healing in a patient by administering a collagen-binding synthetic peptidoglycan to the patient are described. Additionally, methods and compositions are described for decreasing scar formation in a patient by administering a collagen-binding synthetic peptidoglycan to the patient.

### CROSS REFERENCE TO RELATED APPLICATIONS

This application claims priority under 35 U.S.C. §119(e) to U.S. Provisional Application No. 61/175,200, filed May 4, 2009, which is expressly incorporated by reference herein.

### TECHNICAL FIELD

This invention relates to the field of collagen-binding synthetic peptidoglycans. More particularly, this invention relates to collagen-binding synthetic peptidoglycans for use in promoting wound healing and decreasing scar formation in a patient.

### BACKGROUND AND SUMMARY OF THE INVENTION

Collagen is the most abundant protein in the body, presenting many biological signals and maintaining the mechanical integrity of many different tissues. Its molecular organization determines its function, which has made collagen fibrillogenesis a topic of interest in many research fields. Collagen has the ability to self-associate in vitro, forming gels that can act as a 3-dimensional substrate, and provide mechanical and biological signals for cell growth. Research on collagen fibrillogenesis with and without additional extracellular matrix components has raised many questions about the interplay between collagen and other extracellular matrix molecules. There are more than 20 types of collagen currently identified, with type I being the most common. Many tissues are composed primarily of type I collagen including tendon, ligament, skin, and bone. While each of these structures also contains other collagen types, proteoglycans and glycosaminoglycans, and minerals in the case of bone, the principle component is type I collagen. The dramatic difference in mechanical integrity each of these structures exhibits is largely due to the intricate organization of collagen and the interplay with other non-collagen type I components.

Decorin is a proteoglycan that is known to influence collagen fibrillogenesis, which consequently can modify the mechanical and biological information in a collagen gel. The signals resulting from structural changes in collagen organization, as well as the unique signals contained in the glycosaminoglycan chains that are part of proteoglycans, alter cellular behavior and offer a mechanism to design collagen matrices to provide desired cellular responses. Consequently, the Applicants have developed collagen-binding synthetic peptidoglycans which influence collagen organization at the molecular level. These collagen-binding synthetic peptidoglycans are designed based on collagen binding peptides attached to, for example, a glycan, such as a glycosaminoglycan or a polysaccharide, and can be tailored with respect to these components for specific applications. The collagen-binding synthetic peptidoglycans described herein influence the morphological, mechanical, and biological characteristics of collagen matrices, and consequently alter cellular behavior, making these molecules useful for tissue engineering applications.

In one embodiment, a method of promoting wound healing in a patient is described. The method comprises the steps of administering to the patient a collagen-binding synthetic peptidoglycan, wherein the collagen-binding synthetic peptidoglycan promotes healing of a wound in the patient.

In the above described embodiment, the following features, or any combination thereof, apply. In the above described embodiment, 1) the collagen-binding synthetic peptidoglycan can be administered in combination with an excipient selected from the group consisting of hyaluronic acid, poloxamers, collagen, hydroxy methyl cellulose, hydroxy ethyl cellulose, and combinations thereof; 2) the collagen-binding synthetic peptidoglycan can be in the form of an engineered collagen matrix wherein the collagen-binding synthetic peptidoglycan is incorporated into the engineered collagen matrix; 3) the collagen can be selected from the group consisting of type I collagen, type II collagen, type III collagen, type IV collagen, and combinations thereof; 4) the engineered collagen matrix can be formed from a collagen solution wherein the amount of collagen in the collagen solution is from about 0.4 mg/mL to about 6 mg/mL; 5) the molar ratio of the collagen to the collagen-binding synthetic peptidoglycan can be from about 1:1 to about 40:1; 6) the collagen can be crosslinked; 7) the collagen can be uncrosslinked; 8) the collagen-binding synthetic peptidoglycan can have amino acid homology with a portion of the amino acid sequence of a proteoglycan or a protein that regulates collagen fibrillogenesis; 9) the collagen-binding synthetic peptidoglycan can have amino acid homology with a portion of a collagen-binding protein that does not regulate collagen fibrillogenesis; 10) the matrix can further comprise an exogenous population of cells; 11) the exogenous population of cells can be selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells; 12) the matrix can further comprise at least one polysaccharide; 13) the collagen-binding synthetic peptidoglycan can be a compound of formula PₙGₓ wherein n is 1 to 10, wherein x is 1 to 10, P is a synthetic peptide of about 5 to about 40 amino acids comprising a sequence of a collagen-binding domain, and G is a glycan; 14) the collagen-binding synthetic peptidoglycan can be a compound of formula (PₙL)ₓG wherein n is 1 to 5, wherein x is 1 to 10, P is a synthetic peptide of about 5 to about 40 amino acids comprising a sequence of a collagen-binding domain, L is a linker, and G is a glycan; 15) the collagen-binding synthetic peptidoglycan can be a compound of formula P(LGₙ)ₓ wherein n is 1 to 5, x is 1 to 10, P is a synthetic peptide of about 5 to about 40 amino acids comprising a sequence of a collagen-binding domain, L is a linker, and G is a glycan; 16) the glycan can be a glycosaminoglycan or a polysaccharide; 17) the synthetic peptide can have amino acid homology with the amino acid sequence of a small leucine-rich proteoglycan; 18) the peptide can comprise an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC; 19) the glycan can be selected from the group consisting of alginate, agarose, dextran, chondroitin, dermatan, dermatan sulfate, heparan, heparin, keratin, and hyaluronan; 20) the glycan can be dermatan sulfate; 21) the peptide can comprise the amino acid sequence RRANAALKAGELYKSILYGC or GELYKSILYGC; 22) the collagen-binding synthetic peptidoglycan can be administered in a dosage form adapted for topical administration; 23) the collagen-binding synthetic peptidoglycan can be administered in a dosage form adapted for intralesional administration; 24) the collagen-binding synthetic peptidoglycan can be administered in a solution comprising hyaluronic acid or a poloxamer; 25) the dosage form can be selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan; and 26) the powder can contain the collagen-binding synthetic peptidoglycan in lyophilized form.

In another embodiment, a method of decreasing scar formation in a patient is described. The method comprises the steps of administering to the patient a collagen-binding synthetic peptidoglycan, wherein the collagen-binding synthetic peptidoglycan decreases scar formation in the patient.

In the above described embodiment, the following features, or any combination thereof, apply. In the above described embodiment, 1) the collagen-binding synthetic peptidoglycan can be administered in combination with an excipient selected from the group consisting of hyaluronic acid, poloxamers, collagen, hydroxy methyl cellulose, hydroxy ethyl cellulose, and combinations thereof; 2) the collagen-binding synthetic peptidoglycan can be in the form of an engineered collagen matrix wherein the collagen-binding synthetic peptidoglycan is incorporated into the engineered collagen matrix; 3) the collagen can be selected from the group consisting of type I collagen, type II collagen, type III collagen, type IV collagen, and combinations thereof; 4) the engineered collagen matrix can be formed from a collagen solution wherein the amount of collagen in the collagen solution is from about 0.4 mg/mL to about 6 mg/mL; 5) the molar ratio of the collagen to the collagen-binding synthetic peptidoglycan can be from about 1:1 to about 40:1; 6) the collagen can be crosslinked; 7) the collagen can be uncrosslinked; 8) the collagen-binding synthetic peptidoglycan can have amino acid homology with a portion of the amino acid sequence of a proteoglycan or a protein that regulates collagen fibrillogenesis; 9) the collagen-binding synthetic peptidoglycan can have amino acid homology with a portion of a collagen-binding protein that does not regulate collagen fibrillogenesis; 10) the matrix can further comprise an exogenous population of cells; 11) the exogenous population of cells can be selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells; 12) the matrix can further comprise at least one polysaccharide; 13) the collagen-binding synthetic peptidoglycan can be a compound of formula PₙGₓ wherein n is 1 to 10, wherein x is 1 to 10, P is a synthetic peptide of about 5 to about 40 amino acids comprising a sequence of a collagen-binding domain, and G is a glycan; 14) the collagen-binding synthetic peptidoglycan can be a compound of formula (PₙL)ₓG wherein n is 1 to 5, wherein x is 1 to 10, P is a synthetic peptide of about 5 to about 40 amino acids comprising a sequence of a collagen-binding domain, L is a linker, and G is a glycan; 15) the collagen-binding synthetic peptidoglycan can be a compound of formula P(LGₙ)ₓ wherein n is 1 to 5, x is 1 to 10, P is a synthetic peptide of about 5 to about 40 amino acids comprising a sequence of a collagen-binding domain, L is a linker, and G is a glycan; 16) the glycan can be a glycosaminoglycan or a polysaccharide; 17) the synthetic peptide can have amino acid homology with the amino acid sequence of a small leucine-rich proteoglycan; 18) the peptide can comprise an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC; 19) the glycan can be selected from the group consisting of alginate, agarose, dextran, chondroitin, dermatan, dermatan sulfate, heparan, heparin, keratin, and hyaluronan; 20) the glycan can be dermatan sulfate; 21) the peptide can comprise the amino acid sequence RRANAALKAGELYKSILYGC or GELYKSILYGC; 22) the collagen-binding synthetic peptidoglycan can be administered in a dosage form adapted for topical administration; 23) the collagen-binding synthetic peptidoglycan can be administered in a dosage form adapted for intralesional administration; 24) the collagen-binding synthetic peptidoglycan can be administered in a solution comprising hyaluronic acid or a poloxamer; 25) the dosage form can be selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan; and 26) the powder can contain the collagen-binding synthetic peptidoglycan in lyophilized form.

In one embodiment, a composition for use in promoting wound healing in a patient is described. The composition comprises a collagen-binding synthetic peptidoglycan.

In the above described embodiment, the following features, or any combination thereof, apply. In the above described embodiment, 1) the composition can further comprise an excipient selected from the group consisting of hyaluronic acid, poloxamers, collagen, hydroxy methyl cellulose, hydroxy ethyl cellulose, and combinations thereof; 2) the collagen-binding synthetic peptidoglycan can be in the form of an engineered collagen matrix wherein the collagen-binding synthetic peptidoglycan is incorporated into the engineered collagen matrix; 3) the collagen can be selected from the group consisting of type I collagen, type II collagen, type III collagen, type IV collagen, and combinations thereof; 4) the engineered collagen matrix can be formed from a collagen solution wherein the amount of collagen in the collagen solution is from about 0.4 mg/mL to about 6 mg/mL; 5) the molar ratio of the collagen to the collagen-binding synthetic peptidoglycan can be from about 1:1 to about 40:1; 6) the collagen can be crosslinked; 7) the collagen can be uncrosslinked; 8) the collagen-binding synthetic peptidoglycan can have amino acid homology with a portion of the amino acid sequence of a proteoglycan or a protein that regulates collagen fibrillogenesis; 9) the collagen-binding synthetic peptidoglycan can have amino acid homology with a portion of a collagen-binding protein that does not regulate collagen fibrillogenesis; 10) the matrix can further comprise an exogenous population of cells; 11) the exogenous population of cells can be selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells; 12) the matrix can further comprise at least one polysaccharide; 13) the collagen-binding synthetic peptidoglycan can be a compound of formula PₙGₓ wherein n is 1 to 10, wherein x is 1 to 10, P is a synthetic peptide of about 5 to about 40 amino acids comprising a sequence of a collagen-binding domain, and G is a glycan; 14) the collagen-binding synthetic peptidoglycan can be a compound of formula (PₙL)ₓG wherein n is 1 to 5, wherein x is 1 to 10, P is a synthetic peptide of about 5 to about 40 amino acids comprising a sequence of a collagen-binding domain, L is a linker, and G is a glycan; 15) the collagen-binding synthetic peptidoglycan can be a compound of formula P(LGₙ)ₓ wherein n is 1 to 5, x is 1 to 10, P is a synthetic peptide of about 5 to about 40 amino acids comprising a sequence of a collagen-binding domain, L is a linker, and G is a glycan; 16) the glycan can be a glycosaminoglycan or a polysaccharide; 17) the synthetic peptide can have amino acid homology with the amino acid sequence of a small leucine-rich proteoglycan; 18) the peptide can comprise an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC; 19) the glycan can be selected from the group consisting of alginate, agarose, dextran, chondroitin, dermatan, dermatan sulfate, heparan, heparin, keratin, and hyaluronan; 20) the glycan can be dermatan sulfate; 21) the peptide can comprise the amino acid sequence RRANAALKAGELYKSILYGC or GELYKSILYGC; 22) the collagen-binding synthetic peptidoglycan can be administered in a dosage form adapted for topical administration; 23) the collagen-binding synthetic peptidoglycan can be administered in a dosage form adapted for intralesional administration; 24) the composition can further comprise hyaluronic acid or a poloxamer; 25) the dosage form can be selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan; 26) the powder can contain the collagen-binding synthetic peptidoglycan in lyophilized form.

In one embodiment, a composition for use in decreasing scar formation in a patient is described. The composition comprises a collagen-binding synthetic peptidoglycan.

In the above described embodiment, the following features, or any combination thereof, apply. In the above described embodiment, 1) the composition can further comprise an excipient selected from the group consisting of hyaluronic acid, poloxamers, collagen, hydroxy methyl cellulose, hydroxy ethyl cellulose, and combinations thereof; 2) the collagen-binding synthetic peptidoglycan can be in the form of an engineered collagen matrix wherein the collagen-binding synthetic peptidoglycan is incorporated into the engineered collagen matrix; 3) the collagen can be selected from the group consisting of type I collagen, type II collagen, type III collagen, type IV collagen, and combinations thereof; 4) the engineered collagen matrix can be formed from a collagen solution wherein the amount of collagen in the collagen solution is from about 0.4 mg/mL to about 6 mg/mL; 5) the molar ratio of the collagen to the collagen-binding synthetic peptidoglycan can be from about 1:1 to about 40:1; 6) the collagen can be crosslinked; 7) the collagen can be uncrosslinked; 8) the collagen-binding synthetic peptidoglycan can have amino acid homology with a portion of the amino acid sequence of a proteoglycan or a protein that regulates collagen fibrillogenesis; 9) the collagen-binding synthetic peptidoglycan can have amino acid homology with a portion of a collagen-binding protein that does not regulate collagen fibrillogenesis; 10) the matrix can further comprise an exogenous population of cells; 11) the exogenous population of cells can be selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells; 12) the matrix can further comprise at least one polysaccharide; 13) the collagen-binding synthetic peptidoglycan can be a compound of formula PₙGₓ wherein n is 1 to 10, wherein x is 1 to 10, P is a synthetic peptide of about 5 to about 40 amino acids comprising a sequence of a collagen-binding domain, and G is a glycan; 14) the collagen-binding synthetic peptidoglycan can be a compound of formula (PₙL)ₓG wherein n is 1 to 5, wherein x is 1 to 10, P is a synthetic peptide of about 5 to about 40 amino acids comprising a sequence of a collagen-binding domain, L is a linker, and G is a glycan; 15) the collagen-binding synthetic peptidoglycan can be a compound of formula P(LGₙ)ₓ wherein n is 1 to 5, x is 1 to 10, P is a synthetic peptide of about 5 to about 40 amino acids comprising a sequence of a collagen-binding domain, L is a linker, and G is a glycan; 16) the glycan can be a glycosaminoglycan or a polysaccharide; 17) the synthetic peptide can have amino acid homology with the amino acid sequence of a small leucine-rich proteoglycan; 18) the peptide can comprise an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC; 19) the glycan can be selected from the group consisting of alginate, agarose, dextran, chondroitin, dermatan, dermatan sulfate, heparan, heparin, keratin, and hyaluronan; 20) the glycan can be dermatan sulfate; 21) the peptide can comprise the amino acid sequence RRANAALKAGELYKSILYGC or GELYKSILYGC; 22) the collagen-binding synthetic peptidoglycan can be administered in a dosage form adapted for topical administration; 23) the collagen-binding synthetic peptidoglycan can be administered in a dosage form adapted for intralesional administration; 24) the composition can further comprise hyaluronic acid or a poloxamer; 25) the dosage form can be selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan; 26) the powder can contain the collagen-binding synthetic peptidoglycan in lyophilized form.

In one embodiment, a use of a composition comprising a collagen-binding synthetic peptidoglycan in the preparation of a medicament for promoting wound healing in a patient is described.

In the above described embodiment, the following features, or any combination thereof, apply. In the above described embodiment, 1) the composition can further comprise an excipient selected from the group consisting of hyaluronic acid, poloxamers, collagen, hydroxy methyl cellulose, hydroxy ethyl cellulose, and combinations thereof; 2) the collagen-binding synthetic peptidoglycan can be in the form of an engineered collagen matrix wherein the collagen-binding synthetic peptidoglycan is incorporated into the engineered collagen matrix; 3) the collagen can be selected from the group consisting of type I collagen, type II collagen, type III collagen, type IV collagen, and combinations thereof; 4) the engineered collagen matrix can be formed from a collagen solution wherein the amount of collagen in the collagen solution is from about 0.4 mg/mL to about 6 mg/mL; 5) the molar ratio of the collagen to the collagen-binding synthetic peptidoglycan can be from about 1:1 to about 40:1; 6) the collagen can be crosslinked; 7) the collagen can be uncrosslinked; 8) the collagen-binding synthetic peptidoglycan can have amino acid homology with a portion of the amino acid sequence of a proteoglycan or a protein that regulates collagen fibrillogenesis; 9) the collagen-binding synthetic peptidoglycan can have amino acid homology with a portion of a collagen-binding protein that does not regulate collagen fibrillogenesis; 10) the matrix can further comprise an exogenous population of cells; 11) the exogenous population of cells can be selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells; 12) the matrix can further comprise at least one polysaccharide; 13) the collagen-binding synthetic peptidoglycan can be a compound of formula PₙGₓ wherein n is 1 to 10, wherein x is 1 to 10, P is a synthetic peptide of about 5 to about 40 amino acids comprising a sequence of a collagen-binding domain, and G is a glycan; 14) the collagen-binding synthetic peptidoglycan can be a compound of formula (PₙL)ₓG wherein n is 1 to 5, wherein x is 1 to 10, P is a synthetic peptide of about 5 to about 40 amino acids comprising a sequence of a collagen-binding domain, L is a linker, and G is a glycan; 15) the collagen-binding synthetic peptidoglycan can be a compound of formula P(LGₙ)ₓ wherein n is 1 to 5, x is 1 to 10, P is a synthetic peptide of about 5 to about 40 amino acids comprising a sequence of a collagen-binding domain, L is a linker, and G is a glycan; 16) the glycan can be a glycosaminoglycan or a polysaccharide; 17) the synthetic peptide can have amino acid homology with the amino acid sequence of a small leucine-rich proteoglycan; 18) the peptide can comprise an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC; 19) the glycan can be selected from the group consisting of alginate, agarose, dextran, chondroitin, dermatan, dermatan sulfate, heparan, heparin, keratin, and hyaluronan; 20) the glycan can be dermatan sulfate; 21) the peptide can comprise the amino acid sequence RRANAALKAGELYKSILYGC or GELYKSILYGC; 22) the collagen-binding synthetic peptidoglycan can be administered in a dosage form adapted for topical administration; 23) the collagen-binding synthetic peptidoglycan can be administered in a dosage form adapted for intralesional administration; 24) the composition can further comprise hyaluronic acid or a poloxamer; 25) the dosage form can be selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan; 26) the powder can contain the collagen-binding synthetic peptidoglycan in lyophilized form.

In one embodiment, a use of a composition comprising a collagen-binding synthetic peptidoglycan in the preparation of a medicament for decreasing scar formation in a patient is described.

In the above described embodiment, the following features, or any combination thereof, apply. In the above described embodiment, 1) the composition can further comprise an excipient selected from the group consisting of hyaluronic acid, poloxamers, collagen, hydroxy methyl cellulose, hydroxy ethyl cellulose, and combinations thereof; 2) the collagen-binding synthetic peptidoglycan can be in the form of an engineered collagen matrix wherein the collagen-binding synthetic peptidoglycan is incorporated into the engineered collagen matrix; 3) the collagen can be selected from the group consisting of type I collagen, type II collagen, type III collagen, type IV collagen, and combinations thereof; 4) the engineered collagen matrix can be formed from a collagen solution wherein the amount of collagen in the collagen solution is from about 0.4 mg/mL to about 6 mg/mL; 5) the molar ratio of the collagen to the collagen-binding synthetic peptidoglycan can be from about 1:1 to about 40:1; 6) the collagen can be crosslinked; 7) the collagen can be uncrosslinked; 8) the collagen-binding synthetic peptidoglycan can have amino acid homology with a portion of the amino acid sequence of a proteoglycan or a protein that regulates collagen fibrillogenesis; 9) the collagen-binding synthetic peptidoglycan can have amino acid homology with a portion of a collagen-binding protein that does not regulate collagen fibrillogenesis; 10) the matrix can further comprise an exogenous population of cells; 11) the exogenous population of cells can be selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells; 12) the matrix can further comprise at least one polysaccharide; 13) the collagen-binding synthetic peptidoglycan can be a compound of formula PₙGₓ wherein n is 1 to 10, wherein x is 1 to 10, P is a synthetic peptide of about 5 to about 40 amino acids comprising a sequence of a collagen-binding domain, and G is a glycan; 14) the collagen-binding synthetic peptidoglycan can be a compound of formula (PₙL)ₓG wherein n is 1 to 5, wherein x is 1 to 10, P is a synthetic peptide of about 5 to about 40 amino acids comprising a sequence of a collagen-binding domain, L is a linker, and G is a glycan; 15) the collagen-binding synthetic peptidoglycan can be a compound of formula P(LGₙ)ₓ wherein n is 1 to 5, x is 1 to 10, P is a synthetic peptide of about 5 to about 40 amino acids comprising a sequence of a collagen-binding domain, L is a linker, and G is a glycan; 16) the glycan can be a glycosaminoglycan or a polysaccharide; 17) the synthetic peptide can have amino acid homology with the amino acid sequence of a small leucine-rich proteoglycan; 18) the peptide can comprise an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC; 19) the glycan can be selected from the group consisting of alginate, agarose, dextran, chondroitin, dermatan, dermatan sulfate, heparan, heparin, keratin, and hyaluronan; 20) the glycan can be dermatan sulfate; 21) the peptide can comprise the amino acid sequence RRANAALKAGELYKSILYGC or GELYKSILYGC; 22) the collagen-binding synthetic peptidoglycan can be administered in a dosage form adapted for topical administration; 23) the collagen-binding synthetic peptidoglycan can be administered in a dosage form adapted for intralesional administration; 24) the composition can further comprise hyaluronic acid or a poloxamer; 25) the dosage form can be selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan; 26) the powder can contain the collagen-binding synthetic peptidoglycan in lyophilized form.

### The Following Various Embodiments are Provided:

1) A method of promoting wound healing in a patient is described. The method comprises the step of administering to the patient a collagen-binding synthetic peptidoglycan, wherein the collagen-binding synthetic peptidoglycan promotes healing of a wound in the patient.

2) The method of clause 1 wherein the collagen-binding synthetic peptidoglycan is administered in combination with an excipient selected from the group consisting of hyaluronic acid, poloxamers, collagen, hydroxy methyl cellulose, hydroxy ethyl cellulose, and combinations thereof.

3) The method of clause 1 to 2 wherein the collagen-binding synthetic peptidoglycan is in the form of an engineered collagen matrix wherein the collagen-binding synthetic peptidoglycan is incorporated into the engineered collagen matrix.

4) The method of clause 2 to 3 wherein the collagen is selected from the group consisting of type I collagen, type II collagen, type III collagen, type IV collagen, and combinations thereof.

5) The method of clause 3 to 4 wherein the engineered collagen matrix is formed from a collagen solution, and wherein the amount of collagen in the collagen solution is from about 0.4 mg/mL to about 6 mg/mL.

6) The method of clause 2 to 5 wherein the molar ratio of the collagen to the collagen-binding synthetic peptidoglycan is from about 1:1 to about 40:1.

7) The method of clause 2 to 6 wherein the collagen is crosslinked.

8) The method of clause 2 to 6 wherein the collagen is uncrosslinked.

9) The method of clause 1 to 8 wherein the collagen-binding synthetic peptidoglycan has amino acid homology with a portion of the amino acid sequence of a proteoglycan or a protein that regulates collagen fibrillogenesis.

10) The method of clause 1 to 8 wherein the collagen-binding synthetic peptidoglycan has amino acid homology with a portion of a collagen-binding protein that does not regulate collagen fibrillogenesis.

11) The method of clause 3 to 10 wherein the matrix further comprises an exogenous population of cells.

12) The method of clause 11 wherein the exogenous population of cells is selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells.

13) The method of clause 3 to 12 wherein the matrix further comprises at least one polysaccharide.

14) The method of clause 1 to 13 wherein the collagen-binding synthetic peptidoglycan is a compound of formula PₙGₓ wherein n is 1 to 30; wherein x is 1 to 10; P is a synthetic peptide of about 5 to about 40 amino acids comprising a sequence of a collagen-binding domain; and G is a glycan.

15) The method of clause 14 wherein n is 1 to 20.

16) The method of clause 14 to 15 wherein n is 1 to 10.

17) The method of clause 14 to 16 wherein n is 1 to 5.

18) The method of clause 1 to 17 wherein the collagen-binding synthetic peptidoglycan is a compound of formula (PₙL)ₓG wherein n is 1 to 5; wherein x is 1 to 10; P is a synthetic peptide of about 5 to about 40 amino acids comprising a sequence of a collagen-binding domain; L is a linker; and G is a glycan.

19) The method of clause 1 to 17 wherein the collagen-binding synthetic peptidoglycan is a compound of formula P(LGₙ)ₓ wherein n is 1 to 5; x is 1 to 10; P is a synthetic peptide of about 5 to about 40 amino acids comprising a sequence of a collagen-binding domain; L is a linker; and G is a glycan.

20) The method of clause 1 to 19 wherein the glycan is a glycosaminoglycan or a polysaccharide.

21) The method of clause 1 to 20 wherein the synthetic peptide has amino acid homology with the amino acid sequence of a small leucine-rich proteoglycan.

22) The method of clause 1 to 21 wherein the peptide comprises an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC.

23) The method of clause 1 to 22 wherein the glycan is selected from the group consisting of alginate, agarose, dextran, chondroitin, dermatan, dermatan sulfate, heparan, heparin, keratin, and hyaluronan.

24) The method of clause 1 to 23 wherein the glycan is dermatan sulfate.

25) The method of clause 1 to 24 wherein the peptide comprises the amino acid sequence RRANAALKAGELYKSILYGC or GELYKSILYGC.

26) The method of clause 1 to 25 wherein the collagen-binding synthetic peptidoglycan is administered in a dosage form adapted for topical administration.

27) The method of clause 1 to 25 wherein the collagen-binding synthetic peptidoglycan is administered in a dosage form adapted for intralesional administration.

28) The method of clause 1 to 27 wherein the collagen-binding synthetic peptidoglycan is administered in a solution comprising hyaluronic acid or a poloxamer.

29) The method of clause 26 to 28 wherein the dosage form is selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan.

30) The method of clause 29 wherein the powder contains the collagen-binding synthetic peptidoglycan in lyophilized form.

31) A method of decreasing scar formation in a patient is described. The method comprises the step of administering to the patient a collagen-binding synthetic peptidoglycan, wherein the collagen-binding synthetic peptidoglycan decreases scar formation in the patient.

32) The method of clause 31 wherein the collagen-binding synthetic peptidoglycan is administered in combination with an excipient selected from the group consisting of hyaluronic acid, poloxamers, collagen, hydroxy methyl cellulose, hydroxy ethyl cellulose, and combinations thereof.

33) The method of clause 31 to 32 wherein the collagen-binding synthetic peptidoglycan is in the form of an engineered collagen matrix wherein the collagen-binding synthetic peptidoglycan is incorporated into the engineered collagen matrix.

34) The method of clause 32 to 33 wherein the collagen is selected from the group consisting of type I collagen, type II collagen, type III collagen, type IV collagen, and combinations thereof.

35) The method of clause 33 to 34 wherein the engineered collagen matrix is formed from a collagen solution, and wherein the amount of collagen in the collagen solution is from about 0.4 mg/mL to about 6 mg/mL.

36) The method of clause 32 to 35 wherein the molar ratio of the collagen to the collagen-binding synthetic peptidoglycan is from about 1:1 to about 40:1.

37) The method of clause 32 to 36 wherein the collagen is crosslinked.

38) The method of clause 32 to 36 wherein the collagen is uncrosslinked.

39) The method of clause 31 to 38 wherein the collagen-binding synthetic peptidoglycan has amino acid homology with a portion of the amino acid sequence of a proteoglycan or a protein that regulates collagen fibrillogenesis.

40) The method of clause 31 to 38 wherein the collagen-binding synthetic peptidoglycan has amino acid homology with a portion of a collagen-binding protein that does not regulate collagen fibrillogenesis.

41) The method of clause 33 to 40 wherein the matrix further comprises an exogenous population of cells.

42) The method of clause 41 wherein the exogenous population of cells is selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells.

43) The method of clause 33 to 42 wherein the matrix further comprises at least one polysaccharide.

44) The method of clause 31 to 43 wherein the collagen-binding synthetic peptidoglycan is a compound of formula PₙGₓ wherein n is 1 to 30; wherein x is 1 to 10; P is a synthetic peptide of about 5 to about 40 amino acids comprising a sequence of a collagen-binding domain; and G is a glycan.

45) The method of clause 43 wherein n is 1 to 20.

46) The method of clause 44 to 45 wherein n is 1 to 10.

47) The method of clause 44 to 46 wherein n is 1 to 5.

48) The method of clause 31 to 47 wherein the collagen-binding synthetic peptidoglycan is a compound of formula (PₙL)ₓG wherein n is 1 to 5; wherein x is 1 to 10; P is a synthetic peptide of about 5 to about 40 amino acids comprising a sequence of a collagen-binding domain; L is a linker; and G is a glycan.

49) The method of clause 31 to 47 wherein the collagen-binding synthetic peptidoglycan is a compound of formula P(LGₙ)ₓ wherein n is 1 to 5; x is 1 to 10; P is a synthetic peptide of about 5 to about 40 amino acids comprising a sequence of a collagen-binding domain; L is a linker; and G is a glycan.

50) The method of clause 31 to 49 wherein the glycan is a glycosaminoglycan or a polysaccharide.

51) The method of clause 31 to 50 wherein the synthetic peptide has amino acid homology with the amino acid sequence of a small leucine-rich proteoglycan.

52) The method of clause 31 to 51 wherein the peptide comprises an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC.

53) The method of clause 31 to 52 wherein the glycan is selected from the group consisting of alginate, agarose, dextran, chondroitin, dermatan, dermatan sulfate, heparan, heparin, keratin, and hyaluronan.

54) The method of clause 31 to 53 wherein the glycan is dermatan sulfate.

55) The method of clause 31 to 54 wherein the peptide comprises the amino acid sequence RRANAALKAGELYKSILYGC or GELYKSILYGC.

56) The method of clause 31 to 55 wherein the collagen-binding synthetic peptidoglycan is administered in a dosage form adapted for topical administration.

57) The method of clause 31 to 55 wherein the collagen-binding synthetic peptidoglycan is administered in a dosage form adapted for intralesional administration.

58) The method of clause 31 to 57 wherein the collagen-binding synthetic peptidoglycan is administered in a solution comprising hyaluronic acid or a poloxamer.

59) The method of clause 56 to 58 wherein the dosage form is selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan.

60) The method of clause 59 wherein the powder contains the collagen-binding synthetic peptidoglycan in lyophilized form.

61) A composition for use in promoting wound healing in a patient is described, wherein the composition comprises a collagen-binding synthetic peptidoglycan.

62) The composition of clause 61 further comprising an excipient selected from the group consisting of hyaluronic acid, poloxamers, collagen, hydroxy methyl cellulose, hydroxy ethyl cellulose, and combinations thereof.

63) The composition of clause 61 to 62 wherein the collagen-binding synthetic peptidoglycan is in the form of an engineered collagen matrix wherein the collagen-binding synthetic peptidoglycan is incorporated into the engineered collagen matrix.

64) The composition of clause 62 to 63 wherein the collagen is selected from the group consisting of type I collagen, type II collagen, type III collagen, type IV collagen, and combinations thereof.

65) The composition of clause 63 to 64 wherein the engineered collagen matrix is formed from a collagen solution, and wherein the amount of collagen in the collagen solution is from about 0.4 mg/mL to about 6 mg/mL.

66) The composition of clause 62 to 65 wherein the molar ratio of the collagen to the collagen-binding synthetic peptidoglycan is from about 1:1 to about 40:1.

67) The composition of clause 62 to 66 wherein the collagen is crosslinked.

68) The composition of clause 62 to 66 wherein the collagen is uncrosslinked.

69) The composition of clause 61 to 68 wherein the collagen-binding synthetic peptidoglycan has amino acid homology with a portion of the amino acid sequence of a proteoglycan or a protein that regulates collagen fibrillogenesis.

70) The composition of clause 61 to 68 wherein the collagen-binding synthetic peptidoglycan has amino acid homology with a portion of a collagen-binding protein that does not regulate collagen fibrillogenesis.

71) The composition of clause 61 to 70 wherein the matrix further comprises an exogenous population of cells.

72) The composition of clause 71 wherein the exogenous population of cells is selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells.

73) The composition of clause 63 to 72 wherein the matrix further comprises at least one polysaccharide.

74) The composition of clause 61 to 73 wherein the collagen-binding synthetic peptidoglycan is a compound of formula PₙGₓ wherein n is 1 to 30; wherein x is 1 to 10; P is a synthetic peptide of about 5 to about 40 amino acids comprising a sequence of a collagen-binding domain; and G is a glycan.

75) The composition of clause 74 wherein n is 1 to 20.

76) The composition of clause 74 to 75 wherein n is 1 to 10.

77) The composition of clause 74 to 76 wherein n is 1 to 5.

78) The composition of clause 61 to 77 wherein the collagen-binding synthetic peptidoglycan is a compound of formula (PₙL)ₓG wherein n is 1 to 5; wherein x is 1 to 10; P is a synthetic peptide of about 5 to about 40 amino acids comprising a sequence of a collagen-binding domain; L is a linker; and G is a glycan.

79) The composition of clause 61 to 77 wherein the collagen-binding synthetic peptidoglycan is a compound of formula P(LGₙ)ₓ wherein n is 1 to 5; x is 1 to 10; P is a synthetic peptide of about 5 to about 40 amino acids comprising a sequence of a collagen-binding domain; L is a linker; and G is a glycan.

80) The composition of clause 61 to 79 wherein the glycan is a glycosaminoglycan or a polysaccharide.

81) The composition of clause 61 to 80 wherein the synthetic peptide has amino acid homology with the amino acid sequence of a small leucine-rich proteoglycan.

82) The composition of clause 61 to 81 wherein the peptide comprises an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC.

83) The composition of clause 61 to 82 wherein the glycan is selected from the group consisting of alginate, agarose, dextran, chondroitin, dermatan, dermatan sulfate, heparan, heparin, keratin, and hyaluronan.

84) The composition of clause 61 to 83 wherein the glycan is dermatan sulfate.

85) The composition of clause 61 to 84 wherein the peptide comprises the amino acid sequence RRANAALKAGELYKSILYGC or GELYKSILYGC.

86) The composition of clause 61 to 85 wherein the collagen-binding synthetic peptidoglycan is administered in a dosage form adapted for topical administration.

87) The composition of clause 61 to 85 wherein the collagen-binding synthetic peptidoglycan is administered in a dosage form adapted for intralesional administration.

88) The composition of clause 61 to 87 further comprising hyaluronic acid or a poloxamer.

89) The composition of clause 86 to 88 wherein the dosage form is selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan.

90) The composition of clause 89 wherein the powder contains the collagen-binding synthetic peptidoglycan in lyophilized form.

91) A composition for use in decreasing scar formation in a patient is described, wherein the composition comprises a collagen-binding synthetic peptidoglycan.

92) The composition of clause 91 further comprising an excipient selected from the group consisting of hyaluronic acid, poloxamers, collagen, hydroxy methyl cellulose, hydroxy ethyl cellulose, and combinations thereof.

93) The composition of clause 91 to 92 wherein the collagen-binding synthetic peptidoglycan is in the form of an engineered collagen matrix wherein the collagen-binding synthetic peptidoglycan is incorporated into the engineered collagen matrix.

94) The composition of clause 92 to 93 wherein the collagen is selected from the group consisting of type I collagen, type II collagen, type III collagen, type IV collagen, and combinations thereof.

95) The composition of clause 93 to 94 wherein the engineered collagen matrix is formed from a collagen solution, and wherein the amount of collagen in the collagen solution is from about 0.4 mg/mL to about 6 mg/mL.

96) The composition of clause 92 to 95 wherein the molar ratio of the collagen to the collagen-binding synthetic peptidoglycan is from about 1:1 to about 40:1.

97) The composition of clause 92 to 96 wherein the collagen is crosslinked.

98) The composition of clause 92 to 96 wherein the collagen is uncrosslinked.

99) The composition of clause 91 to 98 wherein the collagen-binding synthetic peptidoglycan has amino acid homology with a portion of the amino acid sequence of a proteoglycan or a protein that regulates collagen fibrillogenesis.

100) The composition of clause 91 to 98 wherein the collagen-binding synthetic peptidoglycan has amino acid homology with a portion of a collagen-binding protein that does not regulate collagen fibrillogenesis.

101) The composition of clause 93 to 100 wherein the matrix further comprises an exogenous population of cells.

102) The composition of clause 101 wherein the exogenous population of cells is selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells.

103) The composition of clause 93 to 102 wherein the matrix further comprises at least one polysaccharide.

104) The composition of clause 91 to 103 wherein the collagen-binding synthetic peptidoglycan is a compound of formula PₙGₓ wherein n is 1 to 30; wherein x is 1 to 10; P is a synthetic peptide of about 5 to about 40 amino acids comprising a sequence of a collagen-binding domain; and G is a glycan.

105) The composition of clause 104 wherein n is 1 to 20.

106) The composition of clause 104 to 105 wherein n is 1 to 10.

107) The composition of clause 104 to 106 wherein n is 1 to 5.

108) The composition of clause 91 to 107 wherein the collagen-binding synthetic peptidoglycan is a compound of formula (PₙL)ₓG wherein n is 1 to 5; wherein x is 1 to 10; P is a synthetic peptide of about 5 to about 40 amino acids comprising a sequence of a collagen-binding domain; L is a linker; and G is a glycan.

109) The composition of clause 91 to 107 wherein the collagen-binding synthetic peptidoglycan is a compound of formula P(LGₙ)ₓ wherein n is 1 to 5; x is 1 to 10; P is a synthetic peptide of about 5 to about 40 amino acids comprising a sequence of a collagen-binding domain; L is a linker; and G is a glycan.

110) The composition of clause 91 to 109 wherein the glycan is a glycosaminoglycan or a polysaccharide.

111) The composition of clause 91 to 110 wherein the synthetic peptide has amino acid homology with the amino acid sequence of a small leucine-rich proteoglycan.

112) The composition of clause 91 to 111 wherein the peptide comprises an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC.

113) The composition of clause 91 to 112 wherein the glycan is selected from the group consisting of alginate, agarose, dextran, chondroitin, dermatan, dermatan sulfate, heparan, heparin, keratin, and hyaluronan.

114) The composition of clause 91 to 113 wherein the glycan is dermatan sulfate.

115) The composition of clause 91 to 114 wherein the peptide comprises the amino acid sequence RRANAALKAGELYKSILYGC or GELYKSILYGC.

116) The composition of clause 91 to 115 wherein the collagen-binding synthetic peptidoglycan is administered in a dosage form adapted for topical administration.

117) The composition of clause 91 to 115 wherein the collagen-binding synthetic peptidoglycan is administered in a dosage form adapted for intralesional administration.

118) The composition of clause 91 to 117 further comprising hyaluronic acid or a poloxamer.

119) The composition of clause 116 to 118 wherein the dosage form is selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan.

120) The composition of clause 119 wherein the powder contains the collagen-binding synthetic peptidoglycan in lyophilized form.

121) Use of a composition comprising a collagen-binding synthetic peptidoglycan in the preparation of a medicament for promoting wound healing in a patient is described.

122) The use of clause 121 wherein the composition further comprises an excipient selected from the group consisting of hyaluronic acid, poloxamers, collagen, hydroxy methyl cellulose, hydroxy ethyl cellulose, and combinations thereof.

123) The use of clause 121 to 122 wherein the collagen-binding synthetic peptidoglycan is in the form of an engineered collagen matrix wherein the collagen-binding synthetic peptidoglycan is incorporated into the engineered collagen matrix.

124) The use of clause 122 to 123 wherein the collagen is selected from the group consisting of type I collagen, type II collagen, type III collagen, type IV collagen, and combinations thereof.

125) The use of clause 123 to 124 wherein the engineered collagen matrix is formed from a collagen solution, and wherein the amount of collagen in the collagen solution is from about 0.4 mg/mL to about 6 mg/mL.

126) The use of clause 122 to 125 wherein the molar ratio of the collagen to the collagen-binding synthetic peptidoglycan is from about 1:1 to about 40:1.

127) The use of clause 122 to 126 wherein the collagen is crosslinked.

128) The use of clause 122 to 126 wherein the collagen is uncrosslinked.

129) The use of clause 121 to 128 wherein the collagen-binding synthetic peptidoglycan has amino acid homology with a portion of the amino acid sequence of a proteoglycan or a protein that regulates collagen fibrillogenesis.

130) The use of clause 121 to 128 wherein the collagen-binding synthetic peptidoglycan has amino acid homology with a portion of a collagen-binding protein that does not regulate collagen fibrillogenesis.

131) The use of clause 123 to 130 wherein the matrix further comprises an exogenous population of cells.

132) The use of clause 131 wherein the exogenous population of cells is selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells.

133) The use of clause 123 to 132 wherein the matrix further comprises at least one polysaccharide.

134) The use of clause 121 to 133 wherein the collagen-binding synthetic peptidoglycan is a compound of formula PₙGₓ wherein n is 1 to 30; wherein x is 1 to 10; P is a synthetic peptide of about 5 to about 40 amino acids comprising a sequence of a collagen-binding domain; and G is a glycan.

135) The use of clause 134 wherein n is 1 to 20.

136) The use of clause 134 to 135 wherein n is 1 to 10.

137) The use of clause 134 to 136 wherein n is 1 to 5.

138) The use of clause 121 to 137 wherein the collagen-binding synthetic peptidoglycan is a compound of formula (PₙL)ₓG wherein n is 1 to 5;

wherein x is 1 to 10;

P is a synthetic peptide of about 5 to about 40 amino acids comprising a sequence of a collagen-binding domain;

L is a linker; and

G is a glycan.

139) The use of clause 121 to 137 wherein the collagen-binding synthetic peptidoglycan is a compound of formula P(LGₙ)ₓ wherein n is 1 to 5; x is 1 to 10; P is a synthetic peptide of about 5 to about 40 amino acids comprising a sequence of a collagen-binding domain; L is a linker; and G is a glycan.

140) The use of clause 121 to 139 wherein the glycan is a glycosaminoglycan or a polysaccharide.

141) The use of clause 121 to 140 wherein the synthetic peptide has amino acid homology with the amino acid sequence of a small leucine-rich proteoglycan.

142) The use of clause 121 to 141 wherein the peptide comprises an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC.

143) The use of clause 121 to 142 wherein the glycan is selected from the group consisting of alginate, agarose, dextran, chondroitin, dermatan, dermatan sulfate, heparan, heparin, keratin, and hyaluronan.

144) The use of clause 121 to 143 wherein the glycan is dermatan sulfate.

145) The use of clause 121 to 144 wherein the peptide comprises the amino acid sequence RRANAALKAGELYKSILYGC or GELYKSILYGC.

146) The use of clause 121 to 145 wherein the collagen-binding synthetic peptidoglycan is administered in a dosage form adapted for topical administration.

147) The use of clause 121 to 145 wherein the collagen-binding synthetic peptidoglycan is administered in a dosage form adapted for intralesional administration.

148) The use of clause 121 to 147 wherein the composition further comprises hyaluronic acid or a poloxamer.

149) The use of clause 146 to 148 wherein the dosage form is selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan.

150) The use of clause 149 wherein the powder contains the collagen-binding synthetic peptidoglycan in lyophilized form.

151) Use of a composition comprising a collagen-binding synthetic peptidoglycan in the preparation of a medicament for decreasing scar formation in a patient is described.

152) The use of clause 151 wherein the composition further comprises an excipient selected from the group consisting of hyaluronic acid, poloxamers, collagen, hydroxy methyl cellulose, hydroxy ethyl cellulose, and combinations thereof.

153) The use of clause 151 to 152 wherein the collagen-binding synthetic peptidoglycan is in the form of an engineered collagen matrix wherein the collagen-binding synthetic peptidoglycan is incorporated into the engineered collagen matrix.

154) The use of clause 152 to 153 wherein the collagen is selected from the group consisting of type I collagen, type II collagen, type III collagen, type IV collagen, and combinations thereof.

155) The use of clause 153 to 154 wherein the engineered collagen matrix is formed from a collagen solution, and wherein the amount of collagen in the collagen solution is from about 0.4 mg/mL to about 6 mg/mL.

156) The use of clause 152 to 155 wherein the molar ratio of the collagen to the collagen-binding synthetic peptidoglycan is from about 1:1 to about 40:1.

157) The use of clause 152 to 156 wherein the collagen is crosslinked.

158) The use of clause 152 to 156 wherein the collagen is uncrosslinked.

159) The use of clause 151 to 158 wherein the collagen-binding synthetic peptidoglycan has amino acid homology with a portion of the amino acid sequence of a proteoglycan or a protein that regulates collagen fibrillogenesis.

160) The use of clause 151 to 158 wherein the collagen-binding synthetic peptidoglycan has amino acid homology with a portion of a collagen-binding protein that does not regulate collagen fibrillogenesis.

161) The use of clause 153 to 160 wherein the matrix further comprises an exogenous population of cells.

162) The use of clause 161 wherein the exogenous population of cells is selected from the group consisting of non-keratinized epithelial cells, keratinized epithelial cells, endothelial cells, neural cells, osteoblasts, fibroblasts, chondrocytes, tenocytes, smooth muscle cells, skeletal muscle cells, cardiac muscle cells, progenitor cells, glial cells, synoviocytes, multi-potential progenitor cells, mesodermally derived cells, mesothelial cells, stem cells, and osteogenic cells.

163) The use of clause 153 to 162 wherein the matrix further comprises at least one polysaccharide.

164) The use of clause 151 to 163 wherein the collagen-binding synthetic peptidoglycan is a compound of formula PₙGₓ wherein n is 1 to 30; wherein x is 1 to 10; P is a synthetic peptide of about 5 to about 40 amino acids comprising a sequence of a collagen-binding domain; and G is a glycan.

165) The use of clause 164 wherein n is 1 to 20.

166) The use of clause 164 to 165 wherein n is 1 to 10.

167) The use of clause 164 to 166 wherein n is 1 to 5.

168) The use of clause 151 to 167 wherein the collagen-binding synthetic peptidoglycan is a compound of formula (PₙL)ₓG wherein n is 1 to 5; wherein x is 1 to 10; P is a synthetic peptide of about 5 to about 40 amino acids comprising a sequence of a collagen-binding domain; L is a linker; and G is a glycan.

169) The use of clause 151 to 167 wherein the collagen-binding synthetic peptidoglycan is a compound of formula P(LGₙ)ₓ wherein n is 1 to 5; x is 1 to 10; P is a synthetic peptide of about 5 to about 40 amino acids comprising a sequence of a collagen-binding domain; L is a linker; and G is a glycan.

170) The use of clause 151 to 169 wherein the glycan is a glycosaminoglycan or a polysaccharide.

171) The use of clause 151 to 170 wherein the synthetic peptide has amino acid homology with the amino acid sequence of a small leucine-rich proteoglycan.

172) The use of clause 151 to 171 wherein the peptide comprises an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, and GSITTIDVPWNVGC.

173) The use of clause 151 to 172 wherein the glycan is selected from the group consisting of alginate, agarose, dextran, chondroitin, dermatan, dermatan sulfate, heparan, heparin, keratin, and hyaluronan.

174) The use of clause 151 to 173 wherein the glycan is dermatan sulfate.

175) The use of clause 151 to 174 wherein the peptide comprises the amino acid sequence RRANAALKAGELYKSILYGC or GELYKSILYGC.

176) The use of clause 151 to 175 wherein the collagen-binding synthetic peptidoglycan is administered in a dosage form adapted for topical administration.

177) The use of clause 151 to 175 wherein the collagen-binding synthetic peptidoglycan is administered in a dosage form adapted for intralesional administration.

178) The use of clause 151 to 177 wherein the composition further comprises hyaluronic acid or a poloxamer.

179) The use of clause 176 to 178 wherein the dosage form is selected from the group consisting of a powder, a gel, a cream, a paste, an ointment, a plaster, a lotion, a topical liquid, a bandage impregnated with the collagen-binding synthetic peptidoglycan, and a transdermal patch impregnated with the collagen-binding synthetic peptidoglycan.

180) The use of clause 179 wherein the powder contains the collagen-binding synthetic peptidoglycan in lyophilized form.

### BRIEF DESCRIPTION OF THE DRAWINGS

FIG. 1. shows a schematic representation of the inhibition of lateral aggregation of collagen fibrils by bound peptidoglycan, which is important in determining the mechanical and alignment properties of collagen matrices.

FIG. 2. shows Surface Plasmon Resonance scan in association mode and dissociation mode of peptide RRANAALKAGELYKSILYGC(SILY) binding to collagen bound to CM-3 plates. SILY was dissolved in 1×HBS-EP buffer at varying concentrations from 100 μM to 1.5 μm in 2-fold dilutions.

FIG. 3. shows binding of dansyl-modified peptide SILY to collagen measured in 96-well high-binding plate (black with a clear bottom (Costar)). PBS, buffer only; BSA, BSA-treated well; Collagen, collagen-treated well. Fluorescence readings were taken on an M5 Spectramax Spectrophotometer (Molecular Devices) at excitation/emission wavelengths of 335 nm/490 nm, respectively.

FIG. 4. shows collagen-dansyl-modified peptide SILY binding curve derived from fluorescence data described in FIG. 3.

FIG. 5. shows a schematic description of the reagent, PDPH, and the chemistry of the two-step conjugation of a cysteine-containing peptide with an oxidized glycosylaminoglycoside showing the release of 2-pyridylthiol in the final step.

FIG. 6. shows the measurement of absorbance at 343 nm before DTT treatment of oxidized dermatan sulfate conjugated to PDPH, and after treatment with DTT, which releases 2-pyridylthiol from the conjugate. The measurements allow determination of the ratio of PDPH to oxidized dermatan sulfate. The measured ΔA=0.35, corresponds to 1.1 PDPH molecules/DS.

FIG. 7. shows binding of dansyl-modified peptide SILY conjugated to dermatan sulfate as described herein to collagen measured in 96-well high-binding plate (black with a clear bottom (Costar)). PBS, buffer only; BSA, BSA-treated well; Collagen, collagen-treated well. Fluorescence readings were taken on an M5 Spectramax Spectrophotometer (Molecular Devices) at excitation/emission wavelengths of 335 nm/490 nm, respectively.

FIG. 8. shows the measurement of Shear modulus of gel samples (4 mg/mL collagen, 10:1 collagen:treatment) on a AR-G2 rheometer with 20 mm stainless steel parallel plate geometry (TA Instruments, New Castle, Del.), and the 20 mm stainless steel parallel plate geometry was lowered to a gap distance of 600 μm using a normal force control of 0.25N. Collagen, i.e. collagen alone; DS, collagen+dermatan sulfate; Decorin, collagen+decorin; dermatan sulfate-SILY conjugate, collagen+DS-SILY peptidoglycan; SILY, collagen+RRANAALKAGELYKSILYGC(SILY) peptide. In Panels A., B., and C., treatments added to collagen in a 10:1, 30:1, or 5:1 molar ratio of collagen:treatment, respectively.

FIG. 9. shows the measurement of Shear modulus of gel samples (1.5 mg/mL collagen III, 5:1 collagen:treatment) on a AR-G2 rheometer with 20 mm stainless steel parallel plate geometry (TA Instruments, New Castle, Del.), and the 20 mm stainless steel parallel plate geometry was lowered to a gap distance of 500 μm using a normal force control of 0.25N. ♦—no treatment, i.e. collagen III alone; ▪—collagen+dermatan sulfate (1:1); +—collagen+dermatan sulfate (5:1); x—collagen+dermatan sulfate-KELNLVYTGC (DS-KELN) conjugate (1:1); ▴—collagen+dermatan sulfate-KELN conjugate (5:1); —collagen+KELNLVYTGC (KELN) peptide.

FIG. 10. shows the measurement of Shear modulus of gel samples (1.5 mg/mL collagen III, 5:1 collagen:treatment) on a AR-G2 rheometer with 20 mm stainless steel parallel plate geometry (TA Instruments, New Castle, Del.), and the 20 mm stainless steel parallel plate geometry was lowered to a gap distance of 500 μm using a normal force control of 0.25N. ♦—no treatment, i.e. collagen III alone; ▪—collagen+dermatan sulfate (1:1); +—collagen+dermatan sulfate (5:1); x—collagen+dermatan sulfate-GSIT conjugate (DS-GSIT) (1:1); ▴—collagen+dermatan sulfate-GSIT conjugate (5:1); —collagen+GSITTIDVPWNVGC (GSIT) peptide.

FIG. 11. shows a turbidity measurement. Gel solutions were prepared as described in EXAMPLE 16 (collagen 4 mg/mL and 10:1 collagen to treatment, unless otherwise indicated) and 50 μL/well were added at 4° C. to a 384-well plate. The plate was kept at 4° C. for 4 hours before initiating fibril formation. A SpectraMax M5 at 37° C. was used to measure absorbance at 313 nm at 30 s intervals for 6 hours. Col, no treatment, i.e., collagen alone; DS, collagen+dermatan sulfate; decorin, collagen+decorin; DS-SILY, collagen+dermatan sulfate-SILY conjugate; SILY, collagen+RRANAALKAGELYKSILYGC (SILY) peptide. In Panels A. and B., treatments added at a 10:1 or 1:1 molar ratio of collagen:treatment, respectively.

FIG. 12. shows a turbidity measurement. Gel solutions were prepared as described in EXAMPLE 16 (collagen 4 mg/mL and 1:1 or 10:1 collagen to treatment, unless otherwise indicated) and 50 μL/well were added at 4° C. to a 384-well plate. The plate was kept at 4° C. for 4 hours before initiating fibril formation. A SpectraMax M5 at 37° C. was used to measure absorbance at 313 nm at 30 s intervals for 6 hours. Col, no treatment, i.e., collagen alone; DS, collagen+dermatan sulfate; DS-SILY, collagen+dermatan sulfate-SILY conjugate; DS-Dc13, collagen+dermatan sulfate-Dc13 conjugate; Dc13, collagen+SYIRIADTNITGC (Dc13) peptide.

FIG. 13. shows confocal reflection microscopy images of gels prepared according to EXAMPLE 16 (4 mg/mL collagen, 10:1 collagen:treatment) recorded with an Olympus FV1000 confocal microscope using a 60×, 1.4 NA water immersion lens. Samples were illuminated with 488 nm laser light and the reflected light was detected with a photomultiplier tube using a blue reflection filter. Each gel was imaged 100 μM from the bottom of the gel, and three separate locations were imaged to ensure representative sampling. Collagen, no treatment, i.e., collagen alone; DS, collagen+dermatan sulfate; Decorin, collagen+decorin; Col+DS-SILY, collagen+dermatan sulfate-SILY conjugate.

FIG. 14. shows cryo-scanning electron microscopy images of gel structure at a magnification of 20000, scale bars=4 μm. Gels for cryo-SEM were formed, as in EXAMPLE 16 (4 mg/mL collagen, 10:1 collagen:treatment), directly on the SEM stage and incubated at 37° C. overnight. Each sample evaporated under sublimation conditions for 20 min. The sample was coated by platinum sputter coating for 120 s. Samples were transferred to the cryo-stage at −130° C. and regions with similar orientation were imaged for comparison across treatments. Collagen, no treatment, i.e., collagen alone; Col+DS, collagen+dermatan sulfate; Col+Decorin, collagen+decorin; Col+DS-SILY, collagen+dermatan sulfate-SILY conjugate; Col+DS-SYIR, collagen+dermatan sulfate-SYIR conjugate. Fibril diameter distribution were calculated and presented in histograms adjacent the corresponding image.

FIG. 15. shows cryo-scanning electron microscopy images of gel structure at a magnification of 5000. Gels for cryo-SEM were formed, as described in EXAMPLE 22 (1 mg/mL collagen (Type III), 1:1 collagen:treatment), directly on the SEM stage. Regions with similar orientation were imaged for comparison across treatments. Panel a, Collagen, no treatment, i.e., collagen alone; Panel b, collagen+dermatan sulfate; Panel c, collagen+dermatan sulfate-KELN conjugate; Panel d, collagen+dermatan sulfate-GSIT conjugate.

FIG. 16. shows the average void space fraction measured from the Cryo-SEM images shown in FIG. 15. a) Collagen, no treatment, i.e., collagen alone; b) collagen+dermatan sulfate; c) collagen+dermatan sulfate-KELN conjugate; d) collagen+dermatan sulfate-GSIT conjugate. All differences are significant with p=0.05.

FIG. 17. shows the average fibril diameter measured from the Cryo-SEM images shown in FIG. 14. Collagen, no treatment, i.e., collagen alone; Col+DS, collagen+dermatan sulfate; Col+Decorin, collagen+decorin; Col+DS-SILY, collagen+dermatan sulfate-SILY conjugate; Col+DS-SYIR, collagen+dermatan sulfate-SYIR conjugate.

FIG. 18. shows the average fibril diameter measured from the Cryo-SEM images shown in FIG. 14. Collagen, no treatment, i.e., collagen alone; Dc13, collagen+Dc13 peptide; SILY, collagen+SILY peptide.

FIG. 19. shows oxidation and PDPH conjugation to dermatan sulfate. Dermatan sulfate oxidized by sodium meta-periodate at varying concentrations and subsequently conjugated to PDPH. The number of PDPH molecules conjugated to dermatan sulfate was determined by consumption of PDPH as measured by size exclusion chromatography.

FIG. 20. shows oxidation of dextran (70 kDa) and conjugation to PDPH and GSIT peptide. Dextran at 10 mg/mL was oxidized by sodium meta-periodate at varying concentrations and was subsequently conjugated to PDPH. The number of PDPH molecules conjugated to dextran was determined by consumption of PDPH as measured by size exclusion chromatography. Dextran-PDPH conjugate was subsequently conjugated to GSIT peptide and the number of GSIT peptides per dextran was determined by production of pyridine-2-thione as measured by size exclusion chromatography.

FIG. 21. shows DS-SILY conjugation characterization. After 2 hours, a final ΔA₃₄₃ₙₘ corresponded to 1.06 SILY molecules added to each DS molecule. Note, t=0 is an approximate zero time point due to the slight delay between addition of SILY to the DS-PDPH and measurement of the solution at 343 nm.

FIG. 22. shows conjugation of Dc13 to DS. Production of pyridine-2-thione measured by an increase in absorbance at 343 nm indicates 0.99 Dc13 peptides per DS polymer chain.

FIG. 23. shows Microplate Fluorescence Binding of DS-ZDc13 to Collagen. DS-ZDc13 bound specifically to the collagen surface in a dose-dependent manner.

FIG. 24. shows gel compaction. A. and B. Days 3 and 5 respectively: Decorin and peptidoglycans are significant relative to collagen and DS, * indicates significance compared to collagen, ** indicates significance compared to collagen and DS.

FIG. 25. shows the elastin estimate by Fastin Assay. Panel A: DS-SILY significantly increased elastin production over all samples. DS and DS-Dc13 significantly decreased elastin production over collagen. Control samples of collagen gels with no cells showed no elastin production. Panel B: Free peptides resulted in a slight decrease in elastin production compared to collagen, but no points were significant.

FIG. 26. shows fibril density from Cryo-SEM. Fibril density, defined as the ratio of fibril containing area to void space. DS-SILY and free SILY peptide had significantly greater fibril density, while collagen had significantly lower fibril density. DS-Dc13 was not significant compared to collagen.

FIG. 27. shows the storage modulus (G′) of collagen gels. Rheological mechanical testing of collagen gels formed with each additive at Panel A) 5:1, Panel B) 10:1, and Panel C) 30:1, molar ratio of collagen:additive. Frequency sweeps from 0.1 Hz to 1.0 Hz with a controlled stress of 1.0 Pa were performed. G′avg±S.E. are presented.

FIG. 28. shows cell proliferation and cytotoxicity assays. No significant differences were found between all additives in Panel A) CyQuant, Panel B) Live, and Panel C) Dead assays.

FIG. 29. shows Cryo-SEM images for fibril density. Collagen gels formed in the presence of each additive at a 10:1 molar ratio of collagen:additive. Panel A. DS, Decorin, or peptidoglycans. Panel B. Free Peptides. Images are taken at 10,000×, Scale bar=5 μm.

FIG. 30. shows AFM images of collagen gels. Collagen gels were formed in the presence of each additive at a 10:1 molar ratio of collagen:additive. D-banding is observed for all additives. Images are 1 μm².

FIG. 31. shows collagen degradation determined by hydroxyproline. Treatments: Ctrl, no cells added; Col, collagen without added treatment; DS, dermatan sulfate; Decorin; DS-SILY, dermatan sulfate-SILY conjugate; DS-Dc13, dermatan sulfate-Dc13 conjugate; SILY, SILY peptide; Dc13, Dc13 peptide.

FIG. 32. shows histological scoring for inflammatory response. H&E stained skin samples were scored by a pathologist blinded to the treatments as described. No significant differences were observed at any time point, indicating the addition of DS-SILY does not cause an adverse immune response.

FIG. 33. shows scar strength. Ultimate tensile strength was measured on 4 mm skin strips at each time point (n=12). ** The addition of DS-SILY at low (0.125 mg) and high (0.625 mg) doses significantly increased scar strength compared to NT (no treatment) and HA.

FIG. 34. shows scar strength. The addition of collagen-binding peptidoglycan DS-SILY at both low (0.125 mg) and high (0.25 mg) concentrations increased the ultimate tensile strength of the scar. * Significant vs. No Treatment, ** Significant vs. HA.

FIG. 35. shows visible scar length. The addition of DS-SILY significantly improved the visible scar compared to no treatment or HA controls. The decreased visible scar length measured by 5 blinded observers was significant at 21 days for both doses, but the high dose (0.25 mg) was not significant at 28 days compared to HA control. * Significant vs. No Treatment, ** Significant vs. No Treatment and HA. Visual scar length was measured in this study.

FIG. 36. shows representative scar images. Images captured at 21 and 28 days were used to quantify the visible scar length.

FIG. 37. shows TGF-β1 production of human dermal fibroblasts. TGF-β1 was measured in cell medium of fibroblasts cultured on tissue culture polystyrene treated for 48 hours with 1×PBS, no treatment (NT); or 1.4 μM decorin, DS-SILY peptidoglycan, dermatan sulfate (DS), or SILY peptide dissolved in 1×PBS. * indicates significance compared to NT, and ** indicates significance compared to NT and SILY treatments.

FIG. 38. shows representative trichrome stained histology images at 4× magnification illustrating differences in collagen organization and maturity. Panel A: Untreated, Panel B: HA, Panel C: Peptidoglycan (0.125 mg), Panel D: Peptidoglycan (0.625 mg). Arrows indicate the wound area. Peptidoglycan treatments resulted in nearly scar-free healing as noted by the healthy collagen organization and maturity at 21 days post-injury. Untreated and HA control treatment demonstrate characteristic scar tissue marked by immature, densely packed collagen with parallel orientation and higher vascularity.

FIG. 39. shows tissue samples graded for collagen maturity and organization following established methods. A score of 0 indicates normal healthy skin with no scar formation, while a higher score indicates collagen maturity and organization characteristic of scar tissue. Both peptidoglycan treatments resulted in significantly less scar tissue formation compared to untreated wounds. * Denotes significance compared to no treatment at α=0.05.

FIG. 40. shows purification of intermediate product DS-PDPH by size exclusion chromatography (Panel A). The number of PDPH crosslinkers were determined by calculating the area under the excess PDPH curve and correlating to a standard curve for PDPH to determine the amount consumed. Panel B shows purification of intermediate product DS-BMPH by size exclusion chromatography. The number of BMPH crosslinkers were determined by calculating the area under the excess BMPH curve and correlating to a standard curve for BMPH to determine the amount consumed.

FIG. 41. shows the histological evaluation of trichrome stained tissue following the Beausang scoring system. After 28 days post injury, DS-SILY₄ bulked with 30 mg/mL mannitol and delivered in HA showed a significant improvement over untreated wounds.

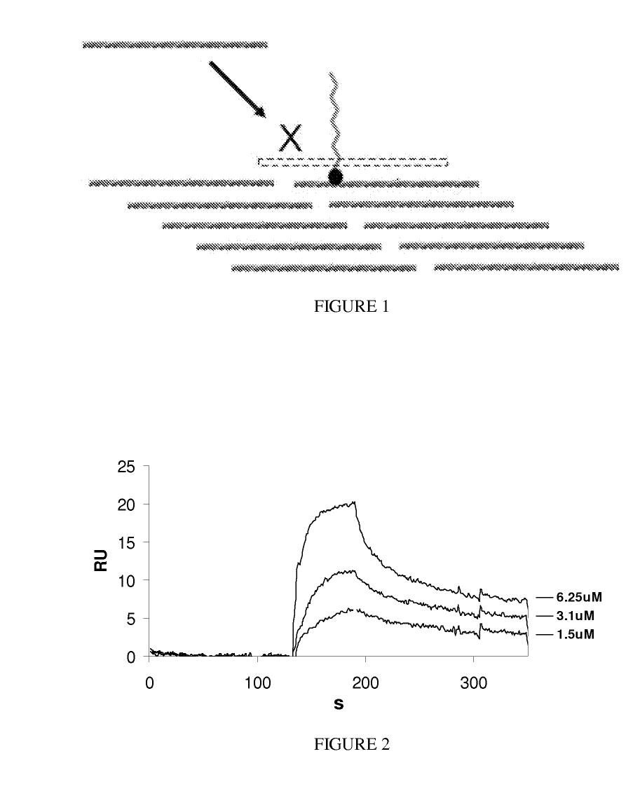

### DETAILED DESCRIPTION OF THE ILLUSTRATIVE EMBODIMENTS

In any of the embodiments described herein, compositions and methods for promoting wound healing in a patient are described. In any of the embodiments described herein, compositions and methods for decreasing scar formation in a patient are described. The compositions comprise a collagen-binding synthetic peptidoglycan for use in promoting wound healing or for use in decreasing scar formation. The methods comprise the step of administering a collagen-binding synthetic peptidoglycan to the patient, and promoting wound healing and/or decreasing scar formation. In any of the various embodiments described herein, the collagen-binding synthetic peptidoglycan is administered in combination with an excipient selected from the group consisting of hyaluronic acid, a poloxamer block polymer, collagen, hydroxy methyl cellulose, hydroxy ethyl cellulose, and combinations thereof. In any of the various embodiments described herein, the collagen-binding synthetic peptidoglycan is incorporated into an engineered collagen matrix and the collagen-binding synthetic peptidoglycan is administered as a component of the engineered collagen matrix.

As used in accordance with this invention, a “collagen-binding synthetic peptidoglycan” means a conjugate of a glycan with a collagen-binding synthetic peptide. The “collagen-binding synthetic peptidoglycans” can have amino acid homology with a portion of a protein or a proteoglycan not normally involved in collagen fibrillogenesis. These collagen-binding synthetic peptidoglycans are referred to herein as “aberrant collagen-binding synthetic peptidoglycans”. The aberrant collagen-binding synthetic peptidoglycans may or may not affect collagen fibrillogenesis. Other collagen-binding synthetic peptidoglycans can have amino acid homology with a portion of a protein or with a proteoglycan normally involved in collagen fibrillogenesis. These collagen-binding synthetic peptidoglycans are referred to herein as “fibrillogenic collagen-binding synthetic peptidoglycans”.

In any of the embodiments described herein, the collagen-binding synthetic peptidoglycans as used herein comprise collagen-binding synthetic peptides of about 5 to about 40 amino acids. In some embodiments, these peptides have homology with the amino acid sequence of a small leucine-rich proteoglycan. In various embodiments, the synthetic peptide comprises an amino acid sequence selected from the group consisting of RLDGNEIKRGC, RRANAALKAGELYKSILYGC, GELYKSILYGC, AHEEISTTNEGVMGC, SQNPVQPGC, NGVFKYRPRYFLYKHAYFYPPLKRFPVQGC, CQDSETRTFY, TKKTLRTGC, GLRSKSKKFRRPDIQYPDATDEDITSHMGC, SYIRIADTNITGC, SYIRIADTNIT, KELNLVYT, KELNLVYTGC, GSITTIDVPWNV, and GSITTIDVPWNVGC. In another embodiment, the synthetic peptide can comprise or can be an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, GSITTIDVPWNVGC, and an amino acid sequence with 80%, 85%, 90%, 95%, or 98% homology with any of these fourteen amino acid sequences. The synthetic peptide is a collagen-binding synthetic peptide.

The glycan (e.g. glycosaminoglycan (GAG) or polysaccharide) attached to the synthetic peptide can be selected from the group consisting of alginate, agarose, dextran, chondroitin, dermatan, dermatan sulfate, heparan, heparin, keratin, and hyaluronan. In one embodiment, the glycan is selected from the group consisting of dermatan sulfate, dextran, and heparin. In another illustrative embodiment, the glycan is dermatan sulfate.

The methods and compositions described herein can be used to treat any condition where the integrity of tissue is damaged, including chronic wounds and acute wounds, wounds in connective tissue, and wounds in muscle, bone and nerve tissue. A “wound”, as used herein includes surgical incisions, burns, acid and alkali burns, cold burn (frostbite), sun burn, ulcers, pressure sores, cuts, abrasions, lacerations, wounds caused by physical trauma, wounds caused by congenital disorders, wounds caused by periodontal disease or following dental surgery, and wounds associated with cancerous tissue or tumors.

As described herein, wounds can include either an acute or a chronic wound. Acute wounds are caused by external damage to intact skin and include surgical wounds, bites, burns, cuts, lacerations, abrasions, etc. Chronic wounds include, for example, those wounds caused by endogenous mechanisms that compromise the integrity of dermal or epithelial tissue, e.g., leg ulcers, foot ulcers, and pressure sores.

In any of the embodiments described herein, the compositions for promoting wound healing or decreasing scar formation may be used at any time to treat chronic or acute wounds. For example, acute wounds associated with surgical incisions can be treated prior to surgery, during surgery, or after surgery to promote wound healing and/or decrease scar formation in a patient. In various illustrative aspects, the compositions as herein described can be administered to the patient in one dose or multiple doses, as necessary to promote wound healing and/or to decrease scar formation.

As used herein, “decreasing scar formation” includes an increase in the ultimate tensile strength of the scar and/or a decrease in the visible scar length. As used herein, a decrease in scar formation also includes complete inhibition of scar formation or complete elimination of visible scarring in a patient.

As used herein, “promoting wound healing” means causing a partial or complete healing of a chronic or an acute wound, or reducing any of the symptoms caused by an acute or a chronic wound. Such symptoms include pain, bleeding, tissue necrosis, tissue ulceration, scar formation, and any other symptom known to result from an acute or a chronic wound.

In any of the embodiments described herein, a method of promoting wound healing is provided. The method comprises the step of administering to the patient a collagen-binding synthetic peptidoglycan, wherein the collagen-binding synthetic peptidoglycan promotes healing of a wound in the patient. In any of the various embodiments described herein, the collagen-binding synthetic peptidoglycan can be an aberrant collagen-binding synthetic peptidoglycan or a fibrillogenic collagen-binding synthetic peptidoglycan with amino acid homology to a portion of the amino acid sequence of a proteoglycan that normally regulates collagen fibrillogenesis.

In any of the embodiments described herein, a method of decreasing scar formation is provided. The method comprises the steps of administering to the patient a collagen-binding synthetic peptidoglycan, wherein the collagen-binding synthetic peptidoglycan decreases scar formation in the patient. In any of the various embodiments described herein, the collagen-binding synthetic peptidoglycan can be an aberrant collagen-binding synthetic peptidoglycan or a fibrillogenic collagen-binding synthetic peptidoglycan with amino acid homology to a portion of the amino acid sequence of a proteoglycan that normally regulates collagen fibrillogenesis.

As discussed above, in any of the embodiments described herein, the collagen-binding synthetic peptidoglycans for use in accordance with the invention comprise peptides of about 5 to about 40 amino acids. In any of the embodiments described herein, the peptide has homology with the amino acid sequence of a small leucine-rich proteoglycan. In various embodiments the synthetic peptide comprises an amino acid sequence selected from the group consisting of RRANAALKAGELYKSILYGC, GELYKSILYGC, RLDGNEIKRGC, AHEEISTTNEGVMGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, NGVFKYRPRYFLYKHAYFYPPLKRFPVQGC, and GSITTIDVPWNVGC. In any of the embodiments described herein, the synthetic peptide can comprise or can be an amino acid sequence selected from the group consisting of RRANAALKAGELYKSILYGC, GELYKSILYGC, RLDGNEIKRGC, AHEEISTTNEGVMGC, NGVFKYRPRYFLYKHAYFYPPLKRFPVQGC, CQDSETRTFY, TKKTLRTGC, GLRSKSKKFRRPDIQYPDATDEDITSHMGC, SQNPVQPGC, SYIRIADTNITGC, SYIRIADTNIT, KELNLVYT, KELNLVYTGC, GSITTIDVPWNV, GSITTIDVPWNVGC, and an amino acid sequence with 80%, 85%, 90%, 95%, or 98% homology to any of these fourteen amino acid sequences.

The glycan attached to the synthetic peptide can be selected from the group consisting of alginate, agarose, dextran, chondroitin, dermatan, dermatan sulfate, heparan, heparin, keratin, and hyaluronan. In any of the embodiments described herein, the glycan is selected from the group consisting of dermatan sulfate, dextran, and heparin. In another illustrative embodiment, the glycan is dermatan sulfate.

In any of the embodiments described herein, the collagen-binding synthetic peptidoglycan can be a compound of any of the following formulas

A) PₙGₓ wherein n is 1 to 10;
wherein x is 1 to 10;
wherein P is a synthetic peptide of about 5 to about 40 amino acids comprising a sequence of a collagen-binding domain; and
wherein G is a glycan.
OR

B) (PₙL)ₓG wherein n is 1 to 5;
wherein x is 1 to 10;
wherein P is a synthetic peptide of about 5 to about 40 amino acids comprising a sequence of a collagen-binding domain;
wherein L is a linker; and
wherein G is a glycan.
OR

C) P(Wₙ)ₓ wherein n is 1 to 5;
wherein x is 1 to 10;
wherein P is a synthetic peptide of about 5 to about 40 amino acids comprising a sequence of a collagen-binding domain;
wherein L is a linker; and
wherein G is a glycan.

In any of the above described formulas, n can be 1 to 5, 1 to 10, 1 to 15, 1 to 20, 1 to 25, or 1 to 30.

In alternative embodiments, a compound of any of the following formulas is provided

A) PₙGₓ wherein n is 1 to 10;
wherein x is 1 to 10;
wherein P is a synthetic peptide of about 5 to about 40 amino acids comprising a sequence of a collagen-binding domain; and
wherein G is a glycan.
OR

B) (PₙL)ₓG wherein n is 1 to 5;
wherein x is 1 to 10;
wherein P is a synthetic peptide of about 5 to about 40 amino acids comprising a sequence of a collagen-binding domain;
wherein L is a linker; and
wherein G is a glycan.
OR

C) P(LGₙ)ₓ wherein n is 1 to 5;
wherein x is 1 to 10;
wherein P is a synthetic peptide of about 5 to about 40 amino acids comprising a sequence of a collagen-binding domain;
wherein L is a linker; and
wherein G is a glycan.

In any of the above described formulas, n can be 1 to 5, 1 to 10, 1 to 15, 1 to 20, 1 to 25, or 1 to 30.

In any of the embodiments described herein, a collagen-binding synthetic peptidoglycan comprising a synthetic peptide of about 5 to about 40 amino acids with amino acid sequence homology to a collagen binding peptide (e.g. a portion of an amino acid sequence of a collagen binding protein or proteoglycan) conjugated to dermatan sulfate, heparin, dextran, or hyaluronan can be used to promote wound healing in a patient, decrease scar formation in a patient, or both. In any of these embodiments, any of the above-described compounds can be used.

In any of the embodiments described herein, the synthetic peptides described herein can be modified by the inclusion of one or more conservative amino acid substitutions. As is well known to those skilled in the art, altering any non-critical amino acid of a peptide by conservative substitution should not significantly alter the activity of that peptide because the side-chain of the replacement amino acid should be able to form similar bonds and contacts as the side chain of the amino acid which has been replaced.

Non-conservative substitutions are possible provided that these do not excessively affect the collagen binding activity of the peptide and/or reduce its effectiveness in promoting wound healing or decreasing scar formation in a patient.

As is well-known in the art, a “conservative substitution” of an amino acid or a “conservative substitution variant” of a peptide refers to an amino acid substitution which maintains: 1) the secondary structure of the peptide; 2) the charge or hydrophobicity of the amino acid; and 3) the bulkiness of the side chain or any one or more of these characteristics. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, ornithine, or histidine. “Negatively charged residues” refer to aspartic acid or glutamic acid. Residues having “bulky side chains” refer to phenylalanine, tryptophan or tyrosine, or the like. A list of illustrative conservative amino acid substitutions is given in TABLE 1.

TABLE 1

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, |
| | D-Gln |
| Aspartic Acid | D-Asp, D-Asn, Asn, Glu, D-Glu, Gln, |
| | D-Gln |
| Cysteine | D-Cys, S-Me-Cys, Met, D-Met, Thr, |
| | D-Thr |
| Glutamine | D-Gln, Asn, D-Asn, Glu, D-Glu, Asp, |
| | D-Asp |
| Glutamic Acid | D-Glu, D-Asp, Asp, Asn, D-Asn, Gln, |
| | D-Gln |
| Glycine | Ala, D-Ala, Pro, D-Pro, Aib, β-Ala |
| Isoleucine | D-Ile, Val, D-Val, Leu, D-Leu, Met, |
| | D-Met |
| Leucine | Val, D-Val, Met, D-Met, D-Ile, |
| | D-Leu, Ile |
| Lysine | D-Lys, Arg, D-Arg, Orn, D-Orn |
| Methionine | D-Met, S-Me-Cys, Ile, D-Ile, Leu, |
| | D-Leu, Val, D-Val |
| Phenylalanine | D-Phe, Tyr, D-Tyr, His, D-His, Trp, |
| | D-Trp |
| Proline | D-Pro |
| Serine | D-Ser, Thr, D-Thr, allo-Thr, L-Cys, |
| | D-Cys |
| Threonine | D-Thr, Ser, D-Ser, allo-Thr, Met, |
| | D-Met, Val, D-Val |
| Tyrosine | D-Tyr, Phe, D-Phe, His, D-His, Trp, |
| | D-Trp |
| Valine | D-Val, Leu, D-Leu, Ile, D-Ile, Met, |
| | D-Met |

In any of the embodiments described herein, a collagen-binding synthetic peptidoglycan comprising a synthetic peptide of about 5 to about 40 amino acids with amino acid sequence homology to a portion of a collagen binding peptide conjugated to dermatan sulfate can be used to promote wound healing or decrease scar formation in a patient. In any of the embodiments described herein, a collagen-binding synthetic peptidoglycan conjugated to dextran can be used to promote wound healing or decrease scar formation in a patient. In any of the embodiments described herein, a collagen-binding synthetic peptidoglycan conjugated to hyaluronan can be used to promote wound healing or decrease scar formation in a patient. In any of these embodiments, any of the above-described compounds can be used.

In any of the embodiments described herein, a collagen-binding synthetic peptidoglycan comprising a synthetic peptide of about 5 to about 40 amino acids with amino acid sequence homology to a collagen binding peptide (e.g. a portion of an amino acid sequence of a collagen binding protein or a proteoglycan) conjugated to any glycan, such as, for example, dermatan sulfate, dextran, or hyaluronan can be used to promote wound healing or decrease scar formation in a patient. In any of these embodiments, any of the above-described compounds can be used.

In any of the embodiments described herein, the synthetic peptide is synthesized according to solid phase peptide synthesis protocols that are well known by persons of skill in the art. In one embodiment a peptide precursor is synthesized on a solid support according to the well-known Fmoc protocol, cleaved from the support with trifluoroacetic acid and purified by chromatography according to methods known to persons skilled in the art.

In any of the embodiments described herein, the synthetic peptide is synthesized utilizing the methods of biotechnology that are well known to persons skilled in the art. In one embodiment a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptide), the plasmid is transfected into a host organism for expression of the peptide, and the peptide is then isolated from the host organism or the growth medium according to methods known by persons skilled in the art (e.g., by affinity column purification). Recombinant DNA technology methods are described in Sambrook et al., “Molecular Cloning: A Laboratory Manual”, 3rd Edition, Cold Spring Harbor Laboratory Press, (2001), incorporated herein by reference, and are well-known to the skilled artisan.

In any of the embodiments described herein, the synthetic peptide is conjugated to a glycan by reacting a free amino group of the peptide with an aldehyde function of the glycan in the presence of a reducing agent, utilizing methods known to persons skilled in the art, to yield the peptide glycan conjugate. In one embodiment an aldehyde function of the glycan (e.g. polysaccharide or glycosaminoglycan) is formed by reacting the glycan with sodium metaperiodate according to methods known to persons skilled in the art.

In any of the embodiments described herein, the synthetic peptide is conjugated to a glycan by reacting an aldehyde function of the glycan with a crosslinker, e.g., 3-(2-pyridyldithio)propionyl hydrazide (PDPH), to form an intermediate glycan and further reacting the intermediate glycan with a peptide containing a free thiol group to yield the peptide glycan conjugate. In any of the various embodiments described herein, the sequence of the peptide may be modified to include a glycine-cysteine segment to provide an attachment point for a glycan or a glycan-linker conjugate. In any of the embodiments described herein, the crosslinker can be N-[β-Maleimidopropionic acid]hydrazide (BMPH).

Although specific embodiments have been described in the preceding paragraphs, the collagen-binding synthetic peptidoglycans described herein can be made by using any art-recognized method for conjugation of the peptide to the glycan (e.g. polysaccharide or glycosaminoglycan). This can include covalent, ionic, or hydrogen bonding, either directly or indirectly via a linking group such as a divalent linker. The conjugate is typically formed by covalent bonding of the peptide to the glycan through the formation of amide, ester or imino bonds between acid, aldehyde, hydroxy, amino, or hydrazo groups on the respective components of the conjugate. All of these methods are known in the art or are further described in the Examples section of this application or in Hermanson G. T., Bioconjugate Techniques, Academic Press, pp. 169-186 (1996), incorporated herein by reference. The linker typically comprises about 1 to about 30 carbon atoms, more typically about 2 to about 20 carbon atoms. Lower molecular weight linkers (i.e., those having an approximate molecular weight of about 20 to about 500) are typically employed.

In any of the embodiments described herein, structural modifications of the linker portion of the conjugates are contemplated. For example, amino acids may be included in the linker and a number of amino acid substitutions may be made to the linker portion of the conjugate, including but not limited to naturally occurring amino acids, as well as those available from conventional synthetic methods. In another aspect, beta, gamma, and longer chain amino acids may be used in place of one or more alpha amino acids. In another aspect, the linker may be shortened or lengthened, either by changing the number of amino acids included therein, or by including more or fewer beta, gamma, or longer chain amino acids. Similarly, the length and shape of other chemical fragments of the linkers described herein may be modified.

In any of the embodiments described herein, the linker may include one or more bivalent fragments selected independently in each instance from the group consisting of alkylene, heteroalkylene, cycloalkylene, cycloheteroalkylene, arylene, and heteroarylene each of which is optionally substituted. As used herein heteroalkylene represents a group resulting from the replacement of one or more carbon atoms in a linear or branched alkylene group with an atom independently selected in each instance from the group consisting of oxygen, nitrogen, phosphorus and sulfur.

In any of the embodiments described herein, a collagen-binding synthetic peptidoglycan may be administered to a patient (e.g., a patient in need of treatment to promote wound healing or decrease scar formation). In any of the various embodiments described herein, routes of administration for the collagen-binding synthetic peptidoglycan can be topical, cutaneous, subcutaneous, percutaneous, intradermal, intraepidermal, intracavernous, intracavitary (e.g., administration within a cavity formed as the result of a wound), intralesional, intramuscular, parenteral, transdermal, or transmucosal, for example. In various illustrative embodiments, the route of administration of the collagen-binding synthetic peptidoglycan can be, for example, via irrigation (e.g., by bathing or flushing an open wound or body cavity), or by an occlusive dressing technique (e.g., by administering the collagen-binding synthetic peptidoglycan via a topical route, then covering the wound with a dressing which occludes the area).

In any of the embodiments described herein, pharmaceutical formulations for use with collagen-binding synthetic peptidoglycans for administration to a patient can comprise: a) a pharmaceutically active amount of the collagen-binding synthetic peptidoglycan; b) a pharmaceutically acceptable pH buffering agent to provide a pH in the range of about pH 4.5 to about pH 9; c) an ionic strength modifying agent in the concentration range of about 0 to about 300 millimolar; and d) an excipient. Any combination of a), b), c) and d) is also provided.

In any of the various embodiments described herein, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES.

In any of the various embodiments described herein, the ionic strength modulating agents include those agents known in the art, for example, glycerin, propylene glycol, mannitol, glucose, dextrose, sorbitol, sodium chloride, potassium chloride, and other electrolytes.

Useful excipients include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxymethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, poloxamer block copolymers (e.g., Pluronic® block copolymers; BASF Corporation, Mount Olive, N.J.), chitosans, gellans or any combination thereof. In one illustrative embodiment, the excipient is collagen. Typically, non-acidic excipients, such as a neutral or basic agent are employed in order to facilitate achieving the desired pH of the formulation. As used herein, the excipient can also act as a viscosity modulating agent.

In any of the embodiments described herein, the excipient can have a concentration ranging from about 0.4 mg/ml to about 6 mg/ml. In various embodiments, the concentration of the excipient may range from about 0.5 mg/ml to about 10 mg/ml, about 0.1 mg/ml to about 6 mg/ml, about 0.5 mg/ml to about 3 mg/ml, about 1 mg/ml to about 3 mg/ml, and about 2 mg/ml to about 4 mg/ml.

In any of the embodiments described herein, suitable formulations may be prepared as a sterile non-aqueous solution or as a dried form to be used in conjunction with a suitable vehicle such as sterile, pyrogen-free water. The preparation of formulations under sterile conditions, for example, by lyophilisation, may readily be accomplished using standard pharmaceutical techniques well known to those skilled in the art.

In any of the embodiments described herein, the solubility of the collagen-binding synthetic peptidoglycan used in the preparation of a suitable formulation may be increased by the use of appropriate formulation techniques, such as the incorporation of solubility-enhancing agents.

In any of the embodiments described herein, suitable formulations may be prepared to be for immediate and/or modified release. Modified release formulations include delayed, sustained, pulsed, controlled, targeted and programmed release formulations. Thus, a collagen-binding synthetic peptidoglycan may be formulated as a solid, semi-solid, or thixotropic liquid for administration as an implanted depot providing modified release of the active compound. Examples of such formulations include copolymeric(dl-lactic, glycolic)acid (PGLA) microspheres. In any of the various embodiments described herein, collagen-binding synthetic peptidoglycans or compositions comprising collagen-binding synthetic peptidoglycans may be continuously administered, where appropriate.

In any of the embodiments described herein, collagen-binding synthetic peptidoglycans and compositions containing them can be administered topically or intralesionally. A variety of dose forms and bases can be used, such as an ointment, cream, gel, gel ointment, paste, plaster (e.g. cataplasm, poultice), lotion, topical liquid, solution, powders, and the like. In any of the various embodiments described herein, the powders can contain the collagen-binding synthetic peptidoglycan in lyophilized form. In one embodiment, a bandage can be impregnated with the collagen-binding synthetic peptidoglycan. In another embodiment, a transdermal patch can be impregnated with the collagen-binding synthetic peptidoglycan. These preparations may be prepared by any conventional method with conventional pharmaceutically acceptable carriers or diluents as described herein.

For example, in the preparation of an ointment, vaseline, higher alcohols, beeswax, vegetable oils, polyethylene glycol, etc. can be used. In the preparation of a cream formulation, fats and oils, waxes, higher fatty acids, higher alcohols, fatty acid esters, purified water, emulsifying agents etc. can be used. In the preparation of gel formulations, conventional gelling materials such as polyacrylates (e.g. sodium polyacrylate), hydroxypropyl cellulose, hydroxymethyl cellulose, hydroxyethyl cellulose, hydroxypropyl methyl cellulose, polyvinyl alcohol, polyvinylpyrrolidone, purified water, lower alcohols, polyhydric alcohols, polyethylene glycol, and the like can be used. In the preparation of a gel ointment preparation, an emulsifying agent (preferably nonionic surfactants), an oily substance (e.g. liquid paraffin, triglycerides, and the like), etc. are used in addition to the gelling materials as mentioned above. A plaster such as cataplasm or poultice can be prepared by spreading a gel preparation as mentioned above onto a support (e.g. fabrics, non-woven fabrics). In addition to the above-mentioned ingredients, paraffins, squalane, lanolin, cholesterol esters, higher fatty acid esters, and the like may optionally be used. Moreover, antioxidants such as BHA, BHT, propyl gallate, pyrogallol, tocopherol, etc. may also be incorporated. In addition to the above-mentioned preparations and components, there may optionally be used any other conventional formulations incorporated with any other suitable additives.

In any of the embodiments described herein, the compositions for promoting wound healing and/or decreasing scar formation can be impregnated into any materials suitable for delivery of the composition to the wound, including cotton, paper, non-woven fabrics, woven fabrics, and knitted fabrics, monofilaments, films, gels, sponges, etc. For example, surgical sutures (monofilaments, twisted yarns or knitting yarns), absorbent pads, transdermal patches, bandages, burn dressings and packings in the form of cotton, paper, non-woven fabrics, woven fabrics, knitted fabrics, films and sponges can be used.

It is also contemplated that any of the formulations described herein may be used to administer the collagen-binding synthetic peptidoglycan (e.g., one or more types) either in the absence or the presence of an engineered collagen matrix as described below.

In any of the various embodiments described herein, the dosage of the collagen-binding synthetic peptidoglycan, with or without an engineered collagen matrix, can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In any of the various embodiments described herein, an effective dose can range from about 1 ng/kg to about 10 mg/kg, 100 ng/kg to about 1 mg/kg, from about 1 μg/kg to about 500 μg/kg, or from about 100 μg/kg to about 400 μg/kg. In each of these embodiments, dose/kg refers to the dose per kilogram of patient mass or body weight. In any of the various embodiments described herein, effective doses can range from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 mg per dose, or from about 500 μg to about 10 mg per dose or from about 1 mg to 10 mg per dose. In other illustrative embodiments, effective doses can be 1 μg, 10 μg, 25 μg, 50 μg, 75 μg, 100 μg, 125 μg, 150 μg, 200 μg, 250 μg, 275 μg, 300 μg, 350 μg, 400 μg, 450 μg, 500 μg, 550 μg, 575 μg, 600 μg, 625 μg, 650 μg, 675 μg, 700 μg, 800 μg, 900 μg, 1.0 mg, 1.5 mg, or 2.0 mg.

Any effective regimen for administering the collagen-binding synthetic peptidoglycan can be used. For example, the collagen-binding synthetic peptidoglycan can be administered as a single dose, or as a multiple-dose daily regimen. Further, a staggered regimen, for example, one to five days per week can be used as an alternative to daily treatment. In any of the various embodiments described herein, the patient is treated with multiple doses of the collagen-binding synthetic peptidoglycan.

In any of the embodiments described herein, a kit or an article of manufacture is provided comprising the collagen-binding synthetic peptidoglycan either alone or in the form of an engineered collagen matrix. The kit or article of manufacture can comprise a container of any type, and the kit or article of manufacture can contain instructions for use of the components of the kit or instructions for use of the article of manufacture. In any of the various embodiments described herein, the components of the kit or article of manufacture are sterilized. The kit or article of manufacture can contain the collagen-binding synthetic peptidoglycan for use as a pharmacological agent.

In any of the embodiments described herein, the kit or article of manufacture can comprise a dose or multiple doses of the collagen-binding synthetic peptidoglycan. In this embodiment, the kit or article of manufacture can further comprise an applicator for manual administration of the collagen-binding synthetic peptidoglycan to the wound. The collagen-binding synthetic peptidoglycan can be in a primary container, for example, a glass vial, such as an amber glass vial with a rubber stopper and/or an aluminum tear-off seal. In another embodiment, the primary container can be plastic or aluminum, and the primary container can be sealed. In another embodiment, the primary container may be contained within a secondary container to further protect the composition from light.

In any of the embodiments described herein, the kit or article of manufacture contains instructions for use. Other suitable kit or article of manufacture components include excipients, disintegrants, binders, salts, local anesthetics (e.g., lidocaine), diluents, preservatives, chelating agents, buffers, tonicity agents, antiseptic agents, wetting agents, emulsifiers, dispersants, stabilizers, and the like. These components may be available separately or admixed with the collagen-binding synthetic peptidoglycan. Any of the composition embodiments described herein can be used to formulate the kit or article of manufacture.

In any of the embodiments described herein, the collagen-binding synthetic peptidoglycan is incorporated into an engineered collagen matrix for administration to the wound or to decrease scar formation. The engineered collagen matrix comprises collagen and a collagen-binding synthetic peptidoglycan. In any of the various embodiments described herein, the engineered collagen matrix may be uncrosslinked. In another embodiment, the matrix may be crosslinked. In any of the various embodiments described herein, crosslinking agents, such as carbodiimides, aldehydes, lysl-oxidase, N-hydroxysuccinimide esters, imidoesters, hydrazides, and maleimides, as well as various natural crosslinking agents, including genipin, and the like, can be added before, during, or after polymerization of the collagen in solution.

As used herein an “engineered collagen matrix” means a collagen matrix where the collagen is polymerized in vitro under predetermined conditions that can be varied and are selected from the group consisting of, but not limited to, pH, phosphate concentration, temperature, buffer composition, ionic strength, and composition and concentration of the collagen.

In any of the embodiments described herein, the collagen used to make the engineered collagen matrix or the collagen for use as excipient may be any type of collagen, including collagen types I to XXVIII, alone or in any combination, for example, collagen types I, II, III, and/or IV may be used. In any of the various embodiments described herein, the engineered collagen matrix is formed using commercially available collagen (e.g., Sigma, St. Louis, Mo.). In any of the various embodiments described herein, the collagen can be purified from submucosa-containing tissue material such as intestinal, urinary bladder, or stomach tissue. In any of the various embodiments described herein, the collagen can be purified from tail tendon. In any of the various embodiments described herein, the collagen can be purified from skin. In any of the various embodiments described herein, the collagen can also contain endogenous or exogenously added non-collagenous proteins in addition to the collagen-binding synthetic peptidoglycans, such as fibronectin, elastin, laminin, fibrin, hyaluronic acid, aggrecan, or silk proteins, glycoproteins, and polysaccharides, or the like. The engineered collagen matrices prepared by the methods described herein can serve as constructs for the regrowth of endogenous tissues at the wound site (e.g., biological remodeling) which can assume the characterizing features of the tissue(s) with which they are associated at the site of implantation or injection into the wound.

In any of the embodiments described herein, either the collagen-binding synthetic peptidoglycan or the engineered collagen matrix containing the collagen-binding synthetic peptidoglycan may be sterilized. As used herein “sterilization” or “sterilize” or “sterilized” means disinfecting by removing unwanted contaminants including, but not limited to, endotoxins, nucleic acid contaminants, and infectious agents.

In any of the various embodiments described herein, either the collagen-binding synthetic peptidoglycan or the engineered collagen matrix containing the collagen-binding synthetic peptidoglycan can be disinfected and/or sterilized using conventional sterilization techniques including glutaraldehyde tanning, formaldehyde tanning at acidic pH, propylene oxide or ethylene oxide treatment, gas plasma sterilization, gamma radiation, electron beam, and/or sterilization with a peracid, such as peracetic acid. Sterilization techniques which do not adversely affect the structure and biotropic properties of the matrix or collagen-binding synthetic peptidoglycan can be used. Illustrative sterilization techniques are exposing the matrix or collagen-binding synthetic peptidoglycan to peracetic acid, 1-4 Mrads gamma irradiation (or 1-2.5 Mrads of gamma irradiation), ethylene oxide treatment, or gas plasma sterilization. In any of the various embodiments described herein, the matrix or collagen-binding synthetic peptidoglycan can be subjected to one or more sterilization processes. In any of the various embodiments described herein, the collagen in solution can also be sterilized or disinfected. The matrix or collagen-binding synthetic peptidoglycan may be wrapped in any type of container including a vial, a plastic wrap or a foil wrap, and may be further sterilized.

In any of the embodiments described herein, the engineered collagen matrix containing the collagen-binding synthetic peptidoglycan may further comprise an added population of cells. The added population of cells may comprise one or more cell populations. In any of the various embodiments described herein, the cell populations comprise a population of non-keratinized or keratinized epithelial cells or a population of cells selected from the group consisting of endothelial cells, mesodermally derived cells, mesothelial cells, synoviocytes, neural cells, glial cells, osteoblasts, fibroblasts, chondrocytes, tenocytes, smooth muscle cells, skeletal muscle cells, cardiac muscle cells, multi-potential progenitor cells (e.g., stem cells, including bone marrow progenitor cells), and osteogenic cells. In various embodiments, combinations of cells can be used.

As discussed above, in accordance with one embodiment, cells can be added to the engineered collagen matrix after polymerization of the collagen or during collagen polymerization. In any of the embodiments described herein, the cells on or within the engineered collagen matrix can be cultured in vitro, for a predetermined length of time, to increase the cell number prior to use in the host.

In any of the embodiments described herein, the compositions described herein can be combined with minerals, amino acids, sugars, peptides, proteins, or laminin, fibronectin, hyaluronic acid, fibrin, elastin, or aggrecan, or growth factors such as epidermal growth factor, platelet-derived growth factor, transforming growth factor beta, or fibroblast growth factor, and glucocorticoids such as dexamethasone or viscoelastic altering agents, such as ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In any of the various embodiments described herein, cross-linking agents, such as carbodiimides, aldehydes, lysl-oxidase, N-hydroxysuccinimide esters, imidoesters, hydrazides, and maleimides, as well as natural crosslinking agents, including genipin.

In any of the embodiments described herein, the collagen solution used to form the engineered collagen matrix can have a collagen concentration ranging from about 0.4 mg/ml to about 6 mg/ml. In various embodiments, the collagen concentration may range from about 0.5 mg/ml to about 10 mg/ml, about 0.1 mg/ml to about 6 mg/ml, about 0.5 mg/ml to about 3 mg/ml, about 1 mg/ml to about 3 mg/ml, and about 2 mg/ml to about 4 mg/ml.

Any of the collagen-binding synthetic peptidoglycans comprising peptides of about 5 to about 40 amino acids described herein can be used to form the engineered collagen matrices in accordance with the invention. Also, any of the glycans described herein can be used including alginate, agarose, dextran, chondroitin, dermatan, dermatan sulfate, heparan, heparin, keratin, and hyaluronan. In any of the embodiments described herein, the glycan is selected from the group consisting of dermatan sulfate, dextran, and heparin. The collagen-binding synthetic peptidoglycan can be lyophilized prior to polymerization, for example, in a buffer or in water or in an acid, such as hydrochloric acid or acetic acid. In any of the various embodiments described herein, the molar ratio of the collagen to the collagen-binding synthetic peptidoglycan can be from about 1:1 to about 40:1.

In any of the embodiments described herein, the polymerizing step can be performed under conditions that are varied where the conditions are selected from the group consisting of pH, phosphate concentration, temperature, buffer composition, ionic strength, the specific components present, and the concentration of the collagen or other components present. In one illustrative aspect, the collagen or other components, including the collagen-binding synthetic peptidoglycan, can be lyophilized prior to polymerization. The collagen or other components can be lyophilized in an acid, such as hydrochloric acid or acetic acid.

In any of the various embodiments described herein, the polymerization reaction is conducted in a buffered solution using any biologically compatible buffer known to those skilled in the art. For example, the buffer may be selected from the group consisting of phosphate buffer saline (PBS), Tris (hydroxymethyl) aminomethane Hydrochloride (Tris-HCl), 3-(N-Morpholino) Propanesulfonic Acid (MOPS), piperazine-n,n′-bis(2-ethanesulfonic acid) (PIPES), [n-(2-Acetamido)]-2-Aminoethanesulfonic Acid (ACES), N-[2-hydroxyethyl]piperazine-N′-[2-ethanesulfonic acid] (HEPES), and 1,3-bis[tris(Hydroxymethyl) methylamino]propane (Bis Tris Propane). In one embodiment the buffer is PBS, Tris, or MOPS and in one embodiment the buffer system is PBS.

In any of the embodiments described herein, the polymerization step is conducted at a pH selected from the range of about 5.0 to about 11, and in one embodiment polymerization is conducted at a pH selected from the range of about 6.0 to about 9.0, and in one embodiment polymerization is conducted at a pH selected from the range of about 6.5 to about 8.5, and in another embodiment the polymerization step is conducted at a pH selected from the range of about 7.0 to about 8.5, and in another embodiment the polymerization step is conducted at a pH selected from the range of about 7.3 to about 7.4.

In any of the various embodiments described herein, the ionic strength of the buffered solution is also regulated. In accordance with one embodiment, the ionic strength of the buffer is selected from a range of about 0.05 to about 1.5 M, in another embodiment the ionic strength is selected from a range of about 0.10 to about 0.90 M, in another embodiment the ionic strength is selected from a range of about 0.14 to about 0.30 M and in another embodiment the ionic strength is selected from a range of about 0.14 to about 0.17 M.

In any of the various embodiments described herein, the polymerization step is conducted at temperatures selected from the range of about 0° C. to about 60° C. In other embodiments, the polymerization step is conducted at temperatures above 20° C., and typically the polymerization is conducted at a temperature selected from the range of about 20° C. to about 40° C., and more typically the temperature is selected from the range of about 30° C. to about 40° C. In one illustrative embodiment the polymerization is conducted at about 37° C.

In any of the various embodiments described herein, the phosphate concentration of the buffer is varied. For example, in one embodiment, the phosphate concentration is selected from a range of about 0.005 M to about 0.5 M. In another illustrative embodiment, the phosphate concentration is selected from a range of about 0.01 M to about 0.2 M. In another embodiment, the phosphate concentration is selected from a range of about 0.01 M to about 0.1 M. In another illustrative embodiment, the phosphate concentration is selected from a range of about 0.01 M to about 0.03 M.

The engineered collagen matrices, including the collagen-binding synthetic peptidoglycans, of the present invention can be combined, prior to, during, or after polymerization, with nutrients, including minerals, amino acids, sugars, peptides, proteins, vitamins (such as ascorbic acid), or other compounds such as laminin and fibronectin, hyaluronic acid, fibrin, elastin, and aggrecan, or growth factors such as epidermal growth factor, platelet-derived growth factor, transforming growth factor beta, vascular endothelial growth factor, or fibroblast growth factor, and glucocorticoids such as dexamethasone, or viscoelastic altering agents, such as ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

In any of the embodiments described herein, cells can be added as the last step prior to the polymerization or after polymerization of the engineered collagen matrix. In any of the various embodiments described herein, cross-linking agents, such as carbodiimides, aldehydes, lysl-oxidase, N-hydroxysuccinimide esters, imidoesters, hydrazides, and maleimides, and the like can be added before, during, or after polymerization.

The matrices can be formed with desired structural, microstructural, nanostructural, or mechanical characteristics. These characteristics can, illustratively, include fibril length, fibril diameter, fibril density, fibril volume fraction, fibril organization, 3-dimensional shape or form, and viscoelastic, tensile, shear, or compressive behavior, permeability, degradation rate, swelling, hydration properties (e.g., rate and swelling), and in vivo tissue remodeling properties, and desired in vivo cell responses. The engineered collagen matrices described herein can have desirable biocompatibility and in vivo remodeling properties, among other desirable or predetermined properties of the matrices incorporating the collagen-binding synthetic peptidoglycans.

As used herein, a “modulus” can be an elastic or linear modulus (defined by the slope of the linear region of the stress-strain curve obtained using conventional mechanical testing protocols; i.e., stiffness), a compressive modulus, a complex modulus, or a shear storage modulus.

As used herein, a “fibril volume fraction” is defined as the percent area of the total area occupied by fibrils in a cross-sectional surface of the matrix in 3 dimensions and “void space fraction” is defined as the percent area of the total area not occupied by fibrils in a cross-sectional surface of the matrix in 3 dimensions.

The engineered collagen matrices described herein comprise collagen fibrils which typically pack in a quarter-staggered pattern giving the fibril a characteristic striated appearance or banding pattern along its axis. In various illustrative embodiments, qualitative and quantitative microstructural characteristics of the engineered collagen matrices can be determined by scanning electron microscopy, transmission electron microscopy, confocal microscopy, second harmonic generation multi-photon microscopy. In another embodiment, tensile, compressive and viscoelastic properties can be determined by rheometry or tensile testing. All of these methods are known in the art or are further described in the Examples section of this application or in Roeder et al., J. Biomech. Eng., vol. 124, pp. 214-222 (2002), in Pizzo et al., J. Appl. Physiol., vol. 98, pp. 1-13 (2004), Fulzele et al., Eur. J. Pharm. Sci., vol. 20, pp. 53-61 (2003), Griffey et al., J. Biomed. Mater. Res., vol. 58, pp. 10-15 (2001), Hunt et al., Am. J. Surg., vol. 114, pp. 302-307 (1967), and Schilling et al., Surgery, vol. 46, pp. 702-710 (1959), incorporated herein by reference.

In any of the various embodiments described herein, the collagen matrices containing collagen-binding synthetic peptidoglycans may be administered to a patient (e.g., a patient in need of treatment to promote wound healing or decrease scar formation in a patient) using any of the formulations, compositions, routes of administration, dosages, or regimens for administration described above for administration of the collagen-binding synthetic peptidoglycan to a patient.

In any of the embodiments herein described, it is to be understood that a combination of two or more collagen-binding synthetic peptidoglycans, differing in the peptide portion, the glycan portion, or both, can be used in place of a single collagen-binding synthetic peptidoglycan.

It is also appreciated that in the foregoing embodiments, certain aspects of the compounds, compositions and methods are presented in the alternative in lists, such as, illustratively, selections for any one or more of G and P. It is therefore to be understood that various alternate embodiments of the invention include individual members of those lists, as well as the various subsets of those lists. Each of those combinations are to be understood to be described herein by way of the lists.

In the following illustrative examples, the terms “synthetic peptidoglycan” and “conjugate” are used synonymously with the term “collagen-binding synthetic peptidoglycan.”

### Example 1

### Peptide Synthesis

All peptides were synthesized using a Symphony peptide synthesizer (Protein Technologies, Tucson, Ariz.), utililizing an FMOC protocol on a Knorr resin. The crude peptide was released from the resin with TFA and purified by reverse phase chromatography on an AKTAexplorer (GE Healthcare, Piscataway, N.J.) utililizing a Grace-Vydac 218TP C-18 reverse phase column and a gradient of water/acetonitrile 0.1% TFA. Dansyl-modified peptides were prepared by adding an additional coupling step with dansyl-Gly (Sigma) before release from the resin. Peptide structures were confirmed by mass spectrometry. The following peptides were prepared as described above: RRANAALKAGELYKSILYGC, SYIRIADTNITGC, Dansyl-GRRANAALKAGELYKSILYGC, and Dansyl-GSYIRIADTNITGC. These peptides are abbreviated SILY, Dc13, ZSILY, and ZDc13. Additional peptides, KELNLVYTGC (abbreviated KELN) and GSITTIDVPWNVGC (abbreviated GSIT) were prepared as described above or purchased (Genescript, Piscataway, N.J.).

### Example 2

### Conjugation of PDPH Peptide to Dermatan Sulfate

The bifunctional crosslinker PDPH (Pierce), reactive to aldehyde and sulfhydryl groups was conjugated to oxDS by a protocol provided by Pierce. PDPH and oxDS (10 mg) was dissolved in 1×PBS pH 7.4 where PDPH was in 10-fold molar excess. The reaction took place at room temperature for 2 hrs protected from light. Excess PDPH was removed by size exclusion chromatography using a HiTrap desalting column (GE Healthcare) equilibrated with MilliQ water. Eluent was monitored at 215 nm, 254 nm, and 280 nm, and consumption of PDPH was measured by integrating the PDPH peak at 215 nm and comparing to a PDPH standard curve generated by desalting varying concentrations of PDPH. The first eluting peak containing DS-PDPH conjugate was collected and lyophilized and stored at −20 C until further testing. Results are presented in FIG. 19 showing controlled oxidation of dermatan sulfate and subsequent conjugation to PDPH.

### Example 3

### Conjugation of SILY to Dermatan Sulfate

The peptide was dissolved in a 5:1 molar excess in coupling buffer at a final peptide concentration of approximately 1 mM (limited by peptide solubility). The reaction was allowed to proceed at room temperature overnight, and excess peptide was separated and the DS-SILY conjugate isolated by size exclusion chromatography as described above. See FIG. 6 showing a SILY/DS ratio of 1.06 after coupling.

### Example 4

### Conjugation of Z-SILY to Dermatan Sulfate

Dermatan sulfate was conjugated to Z-SILY according to the method of EXAMPLE 3.

### Example 5

### Conjugation of KELN to Dermatan Sulfate

Dermatan sulfate was conjugated to KELN according to the method of EXAMPLE 3.

### Example 6

### Conjugation of GSIT to Dermatan Sulfate

Dermatan sulfate was conjugated to GSIT according to the method of EXAMPLE 3.

### Example 7

### Conjugation of Z- of ZDc13 to Dermatan Sulfate

Dermatan sulfate was conjugated to Z-SYIR according to the method of EXAMPLE 2.

### Example 8

### Conjugation of GSIT to Dextran

Dextran (70 kDa), purchased from Sigma-Aldrich was oxidized by sodium meta-periodate oxidation. Dextran (50 mg) was dissolved into 5 mL periodate buffer (0.1M sodium acetate pH 5.5) and varying amounts of sodium meta-periodate were added to the reaction mixture. The reaction took place at room temperature for 30 minutes protected from light forming oxidized dextran (oxDex). Excess sodium meta-periodate was removed by size exclusion chromatography using a HiTrap size exclusion column as described. OxDex was lyophilized and stored at −20 C protected from light until further processing.

OxDex was conjugated to PDPH by the method described for conjugating oxDS to PDPH in EXAMPLE 2. PDPH was reacted in 10 to 20-fold molar excess in 5 mL 1×PBS at room temperature protected from light. Excess PDPH was removed by size exclusion chromatography and the number of PDPH molecules conjugated to dextran was determined by the consumption of PDPH as measured by integration of the PDPH peak at 215 nm. Dex-PDPH was lyophilized and stored at −20 C until further processing.

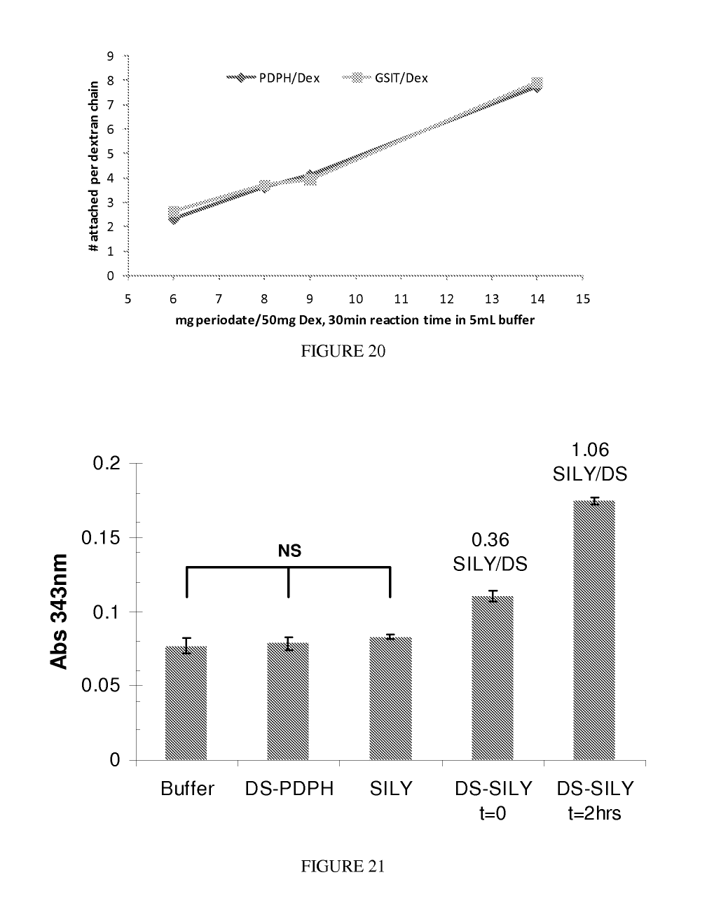

Dex-PDPH was conjugated to GSIT peptide by a similar conjugation protocol as described for DS-SILY in EXAMPLE 3. GSIT was reacted in 10 to 20-fold molar excess in 5 mL 1×PBS pH 7.4 for 4 hours at room temperature. Excess GSIT peptide was removed by size exclusion chromatography using two HiTrap columns in series. Eluent was monitored at 215 nm, 343 nm, and 280 nm. The number of GSIT peptides attached to dextran was determined by quantification of pyridine-2-thione as measured by integrating the pyridine-2-thione peak at 343 nm and determining mass from a pyridine-2-thione standard curve generated by desalting varying amounts of pyridine-2-thione. Controlled oxidation and conjugation of GSIT peptide to dextran was achieved by varying the amount of sodium meta-periodate as shown in FIG. 20.

### Example 9

### Conjugation of GSIT to Heparin

Heparin was conjugated to GSIT according to the method of EXAMPLE 8 (abbreviated Hep-GSIT).

### Example 10

### Conjugation of SILY to Dextran

Dextran was conjugated to SILY according to the method of EXAMPLE 8 replacing heparin with dextran. Modification of the conditions for oxidation of dextran with sodium meta-periodate in the first step to allowed preparation of conjugates with different molar ratios of SILY to dextran. For example dextran-SILY conjugates with a molar ratio of SILY to dextran of about 6 and a dextran-SILY conjugate with a molar ratio of SILY to dextran of about 9 were prepared (abbreviated Dex-SILY6 and Dex-SILY9).

### Example 11

### Conjugation of SILY to Hyaluronan

Hyaluronan was conjugated to SILY according to the method of EXAMPLE 8 (abbreviated HA-SILY).

### Example 12

### SILY Binding to Collagen (Biacore)

Biacore studies were performed on a Biacore 2000 using a CM-3 chip (Biacore, Inc., Piscataway, N.J.). The CM-3 chip is coated with covalently attached carboxymethylated dextran, which allows for attachment of the substrate collagen via free amine groups. Flow cells (FCs) 1 and 2 were used, with FC-1 as the reference cell and FC-2 as the collagen immobilized cell. Each FC was activated with EDC-NHS, and 1500RU of collagen was immobilized on FC-2 by flowing 1 mg/mL collagen in sodium acetate, pH 4, buffer at 5 μL/min for 10 min. Unreacted NHS-ester sites were capped with ethanolamine; the control FC-1 was activated and capped with ethanolamin.

To determine peptide binding affinity, SILY was dissolved in 1×HBS-EP buffer (Biacore) at varying concentrations from 100 uM to 1.5 μm in 2-fold dilutions. The flow rate was held at 90 μL/min which is in the range suggested by Myska for determining binding kinetics (Myska, 1997). The first 10 injections were buffer injections, which help to prime the system, followed by randomized sample injections, run in triplicate. Analysis was performed using BIAevaluation software (Biacore). Representative association/disassociation curves are shown in FIG. 2 demonstrating that the SILY peptide binds reversibly with collagen. K_{D}=1.2 μM was calculated from the on-off binding kinetics.

### Example 13

### Z-SILY Binding to Collagen

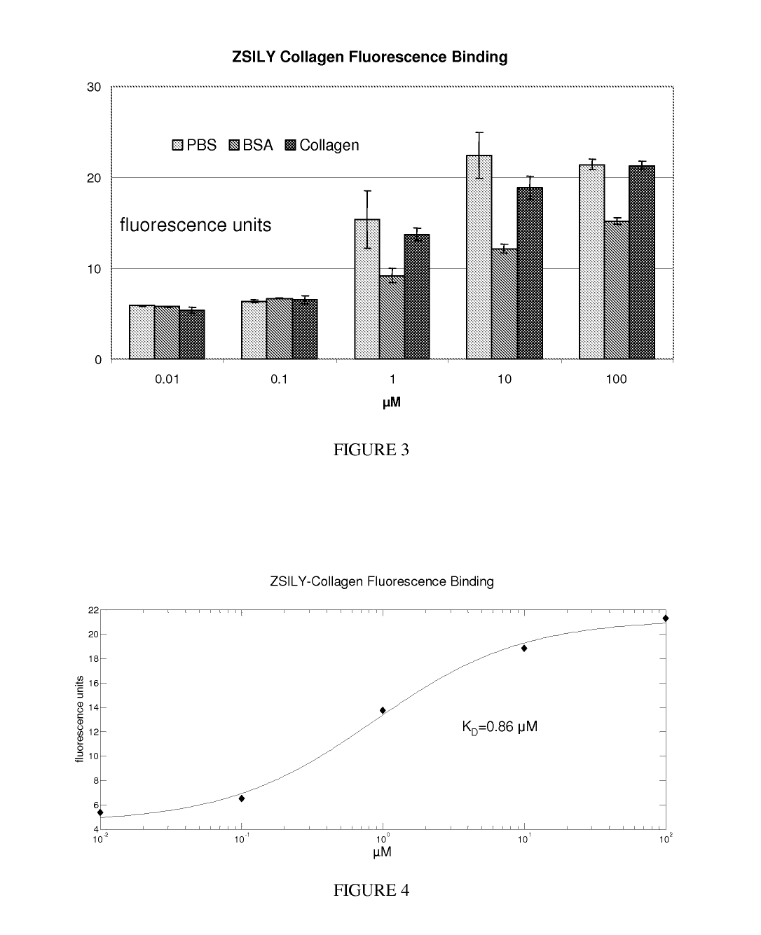

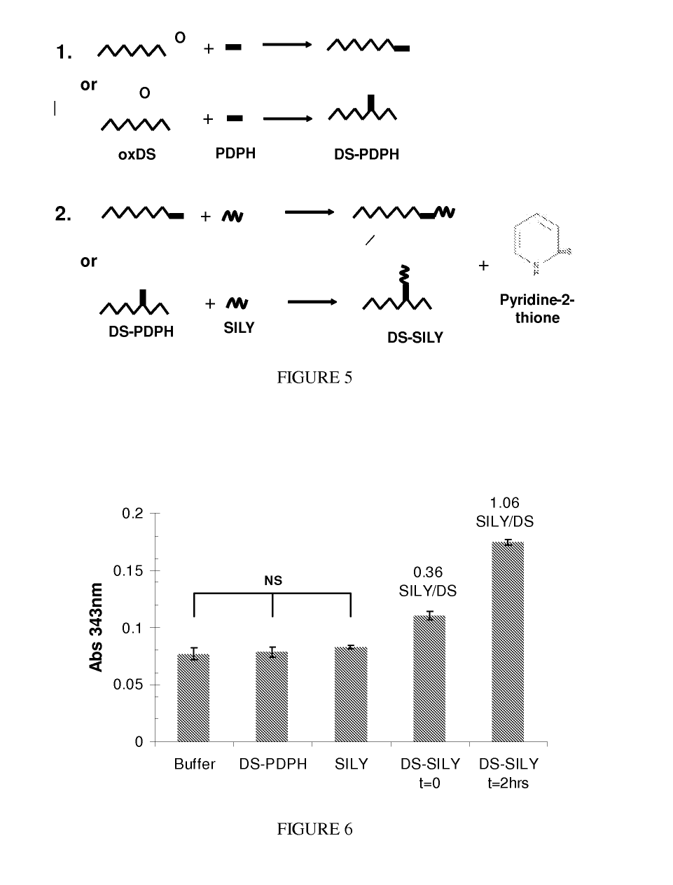

Binding assays were done in a 96-well high-binding plate, black with a clear bottom (Costar). Collagen was compared to untreated wells and BSA coated wells. Collagen and BSA were immobilized at 37° C. for 1 hr by incubating 90 μL/well at concentrations of 2 mg/mL in 10 mM HCl and 1×PBS, respectively. Each well was washed 3× with 1×PBS after incubating. Z-SILY was dissolved in 1×PBS at concentrations from 100 μM to 10 nM in 10-fold dilutions. Wells were incubated for 30 min at 37° C. and rinsed 3× with PBS and then filled with 90 μL of 1×PBS. Fluorescence readings were taken on an M5 Spectramax Spectrophotometer (Molecular Devices) at excitation/emission wavelengths of 335 nm/490 nm respectively. The results are shown in FIGS. 3 and 4. K_{D}=0.86 μM was calculated from the equilibrium kinetics.

### Example 14

### Characterizing DS-SILY

To determine the number of SILY molecules conjugated to DS, the production of pyridine-2-thione was measured using a modified protocol provided by Pierce. Dermatan sulfate with 1.1 PDPH molecules attached was dissolved in coupling buffer (0.1M sodium phosphate, 0.25M sodium chloride) at a concentration of 0.44 mg/mL and absorbance at 343 nm was measured using a SpectraMax M5 (Molecular Devices). SILY was reacted in 5-fold molar excess and absorbance measurements were repeated immediately after addition of SILY and after allowing to react for 2 hours. To be sure SILY does not itself absorb at 343 nm, coupling buffer containing 0.15 mg/mL SILY was measured and was compared to absorbance of buffer alone.

The number of SILY molecules conjugated to DS was calculated by the extinction coefficient of pyridine-2-thione using the following equation (Abs₃₄₃/8080)×(MW_{DS}/DS_{mg/mL}). The results are shown in FIG. 21.

Alternatively, the number of SILY molecules conjugated to DS can be determined by quantifying the pyridine-2-thione peak during size exclusion chromatography, and comparing values to a pyridine-2-thione standard curve generated by desalting varying amounts of pyridine-2-thione.

### Example 15

### Collagen Binding, Fluorescence Data—DS-SILY

In order to determine whether the peptide conjugate maintained its ability to bind to collagen after its conjugation to DS, a fluorescent binding assay was performed. A fluorescently labeled version of SILY, Z-SILY, was synthesized by adding dansylglycine to the amine terminus. This peptide was conjugated to DS and purified using the same methods described for SILY.

Binding assays were done in a 96-well high binding plate, black with a clear bottom (Costar). Collagen was compared to untreated wells and BSA coated wells. Collagen and BSA were immobilized at 37° C. for 1 hr by incubating 90 μL/well at concentrations of 2 mg/mL in 10 mM HCl and 1×PBS respectively. Each well was washed 3× with 1×PBS after incubating.

Wells were preincubated with DS at 37° C. for 30 min to eliminate nonspecific binding of DS to collagen. Wells were rinsed 3× with 1×PBS before incubating with DS-Z-SILY. DS-Z-SILY was dissolved in 1×PBS at concentrations from 100 μM to 10 nM in 10-fold dilutions. Wells were incubated for 30 min at 37° C. and rinsed 3× and then filled with 90 μL of 1×PBS. Fluorescence readings were taken on an M5 Spectramax Spectrophotometer (Molecular Devices) at excitation/emission wavelengths of 335 nm/490 nm respectively.

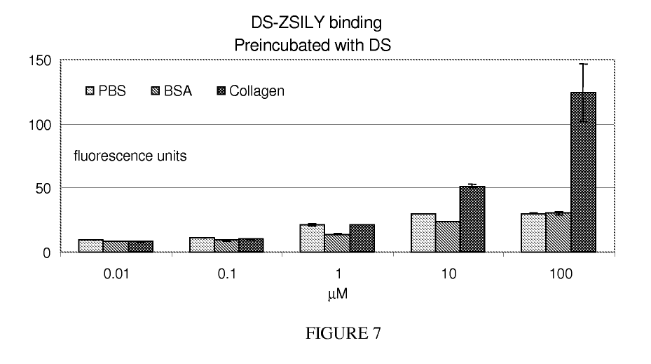

Fluorescence binding of DS-Z-SILY on immobilized collagen, BSA, and untreated wells are compared in FIG. 7. Results show that DS-Z-SILY binds specifically to the collagen-treated wells over BSA and untreated wells. The untreated wells of the high bind plate were designed to be a positive control, though little binding was observed relative to collagen treated wells. These results suggest that SILY maintains its ability to bind to collagen after it is conjugated to DS. Preincubating with DS did not prevent binding, suggesting that the conjugate binds separately from DS alone.

### Example 16

### Preparation of Type I Collagen Gels

Gels were made with Nutragen collagen (Inamed, Freemont, Calif.) at a final concentration of 4 mg/mL collagen. Nutragen stock is 6.4 mg/mL in 10 mM HCl. Gel preparation was performed on ice, and fresh samples were made before each test. The collagen solution was adjusted to physiologic pH and salt concentration, by adding appropriate volumes of 10×PBS (phosphate buffered saline), 1×PBS, and 1M NaOH. For most experiments, samples of DS, decorin, DS-SILY, or DS-Dc13 were added at a 10:1 collagen:treatment molar ratio by a final 1×PBS addition (equal volumes across treatments) in which the test samples were dissolved at appropriate concentrations. In this way, samples are constantly kept at pH 7.4 and physiologic salt concentration. Collagen-alone samples received a 1×PBS addition with no sample dissolved. Fibrillogenesis will be induced by incubating neutralized collagen solutions at 37° C. overnight in a humidified chamber to avoid dehydration. Gel solutions with collagen:treatment molar ratios of other than 10:1 were prepared similarly.

### Example 17

### Viscoelastic Characterization of Gels

Collagen gels were prepared as described in EXAMPLE 16 and prior to heating, 200 μL of each treatment were pipetted onto the wettable surface of hydrophobically printed slides (Tekdon). The PTFE printing restricted gels to the 20 mm diameter wettable region. Gels were formed in a humidified incubator at 37° C. overnight prior to mechanical testing.

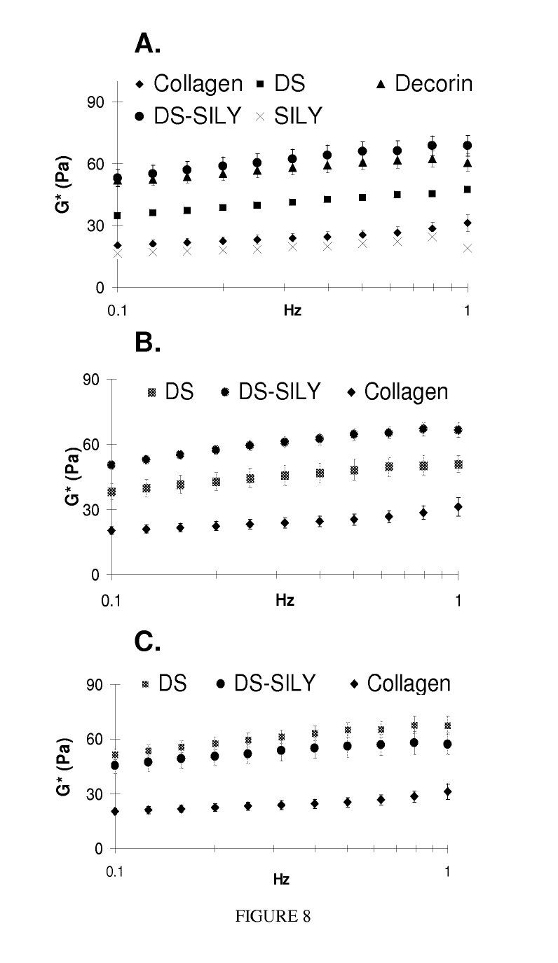

Slides were clamped on the rheometer stage of a AR-G2 rheometer with 20 mm stainless steel parallel plate geometry (TA Instruments, New Castle, Del.), and the 20 mm stainless steel parallel plate geometry was lowered to a gap distance of 600 μm using a normal force control of 0.25N to avoid excessive shearing on the formed gel. An iterative process of stress and frequency sweeps was performed on gels of collagen alone to determine the linear range. All samples were also tested over a frequency range from 0.1 Hz to 1.0 Hz and a controlled stress of 1.0 Pa. Statistical analysis using DesignExpert software (StatEase, Minneapolis, Minn.) was performed at each frequency and a 1-way ANOVA used to compare samples. The results shown in FIG. 8, Panel A. 10:1; Panel B. 30:1, Panel C. 5:1 demonstrate that treatment with synthetic peptidoglycans can modify the viscoelastic behavior of collagen type I gels.

### Example 18

### Viscoelastic Characterization of Collagen III Containing Gels

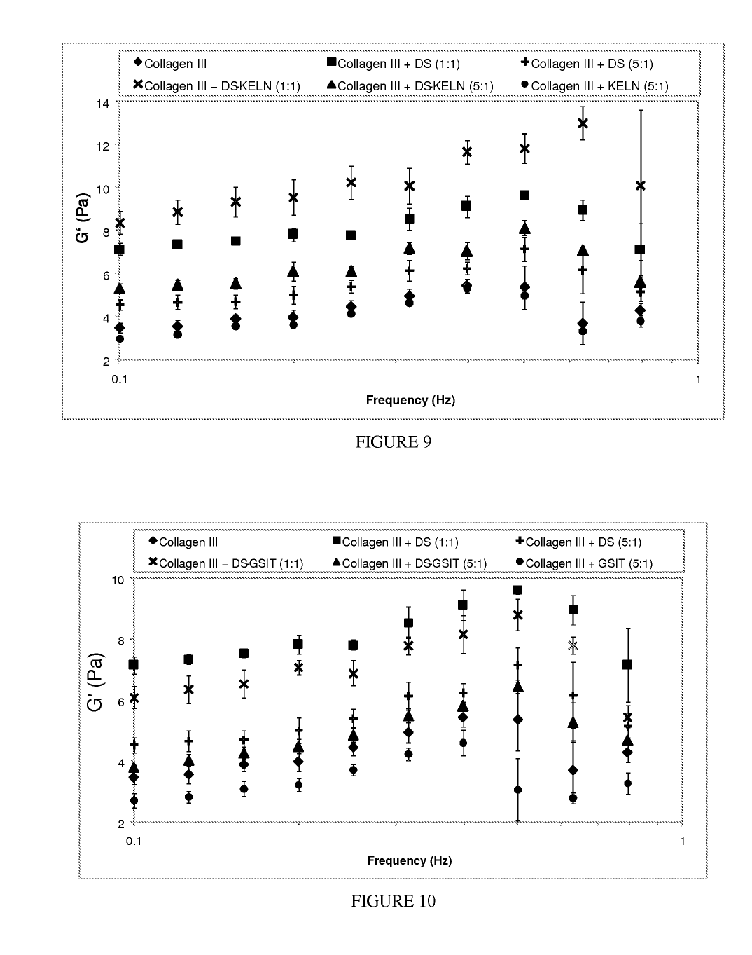

Gels containing type III collagen were prepared as in EXAMPLE 16 with the following modifications: treated and untreated gel solutions were prepared using a collagen concentration of 1.5 mg/mL (90% collagen III (Millipore), 10% collagen I), 200 μL samples were pipetted onto 20 mm diameter wettable surfaces of hydrophobic printed slides. These solutions were allowed to gel at 37° C. for 24 hours. Gels were formed from collagen alone, collagen treated with dermatan sulfate (1:1 and 5:1 molar ratio), and collagen treated with the collagen III-binding peptides alone (GSIT and KELN, 5:1 molar ratio) served as controls. The treated gels contained the peptidoglycans (DS-GSIT or DS-KELN at 1:1 and 5:1 molar ratios. All ratios are collagen:treatment compound ratios. The gels were characterized as in EXAMPLE 17, except the samples were tested over a frequency range from 0.1 Hz to 1.0 Hz at a controlled stress of 1.0 Pa. As shown in FIGS. 9 and 10, the dermatan sulfate-GSIT conjugate and the dermatan sulfate-KELN conjugate (synthetic peptidoglycans) can influence the viscoelastic properties of gels formed with collagen type III.

### Example 19

### Fibrillogenesis

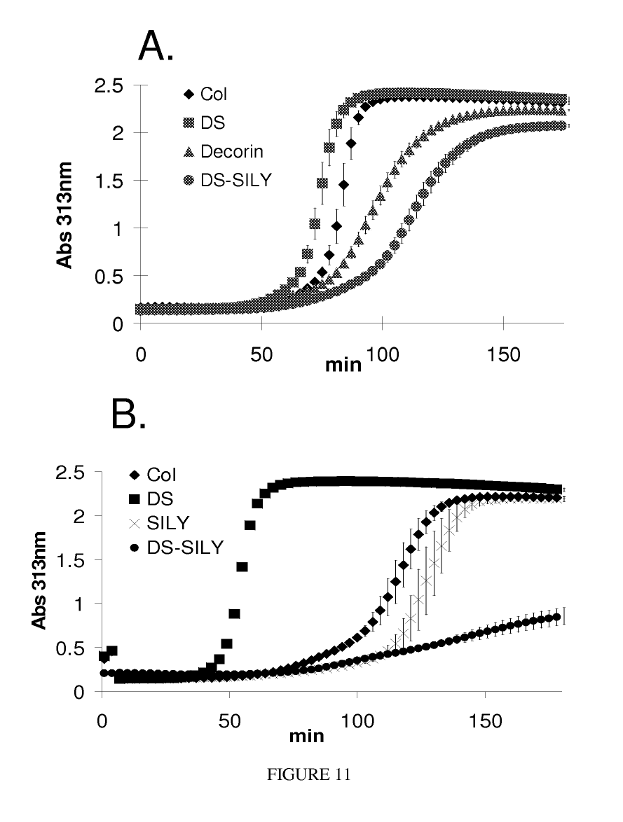

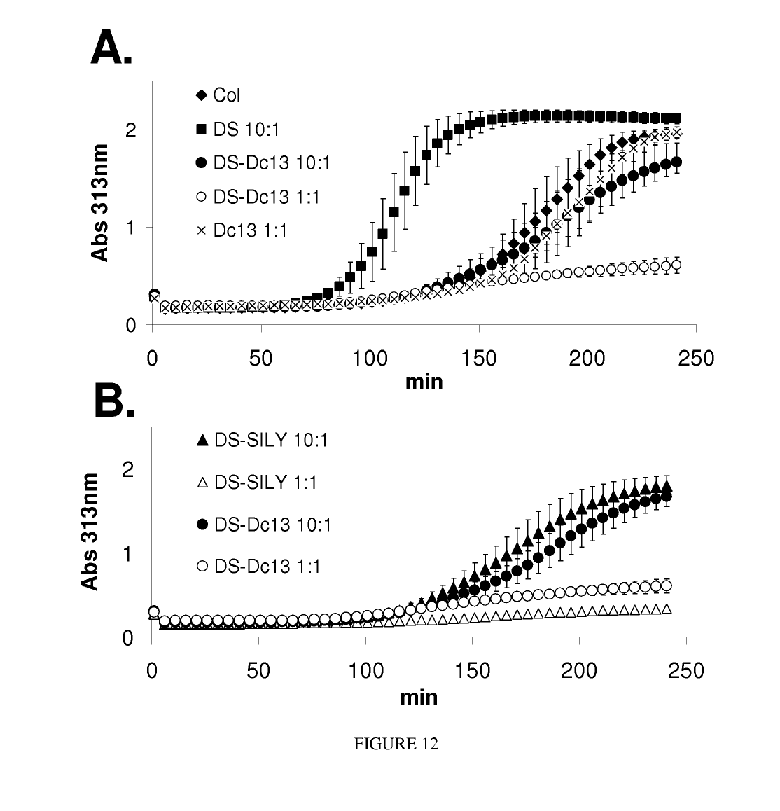

Collagen fibrillogenesis was monitored by measuring turbidity related absorbance at 313 nm providing information on rate of fibrillogenesis and fibril diameter. Gel solutions were prepared as described in EXAMPLE 16 (4 mg/mL collagen, 10:1 collagen:treatment, unless otherwise indicated) and 50 uL/well were added at 4° C. to a 384-well plate. The plate was kept at 4° C. for 4 hours before initiating fibril formation. A SpectraMax M5 at 37° C. was used to measure absorbance at 313 nm at 30 s intervals for 6 hours. The results are shown in FIGS. 11 and 12. Dermatan sulfate-SILY and dermatan sulfate-Dc13 decrease the rate of fibrillogenesis.

### Example 20

### Confocal Reflection Microscopy

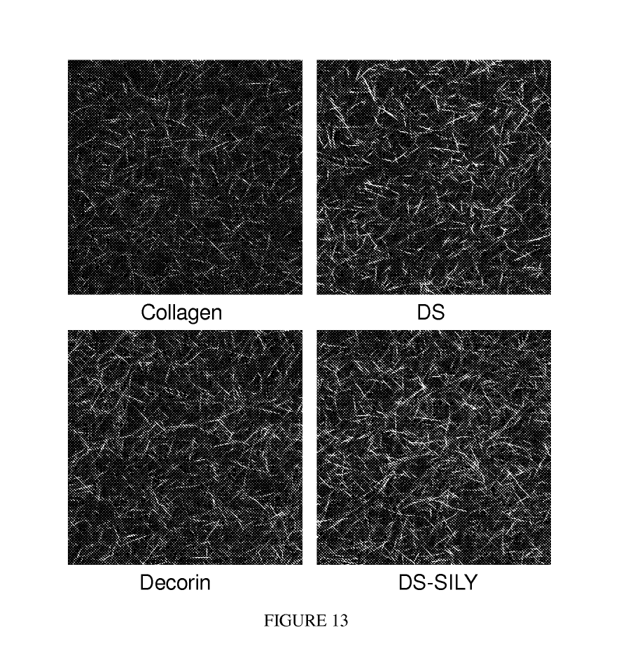

Gels were formed and incubated overnight as described above in EXAMPLE 16, the gels were imaged with an Olympus FV1000 confocal microscope using a 60×, 1.4 NA water immersion lens. Samples were illuminated with 488 nm laser light and the reflected light was detected with a photomultiplier tube using a blue reflection filter. Each gel was imaged 100 μM from the bottom of the gel, and three separate locations were imaged to ensure representative sampling. Results are shown in FIG. 13.

### Example 21

### Cryo-SEM Measurements on Collagen I

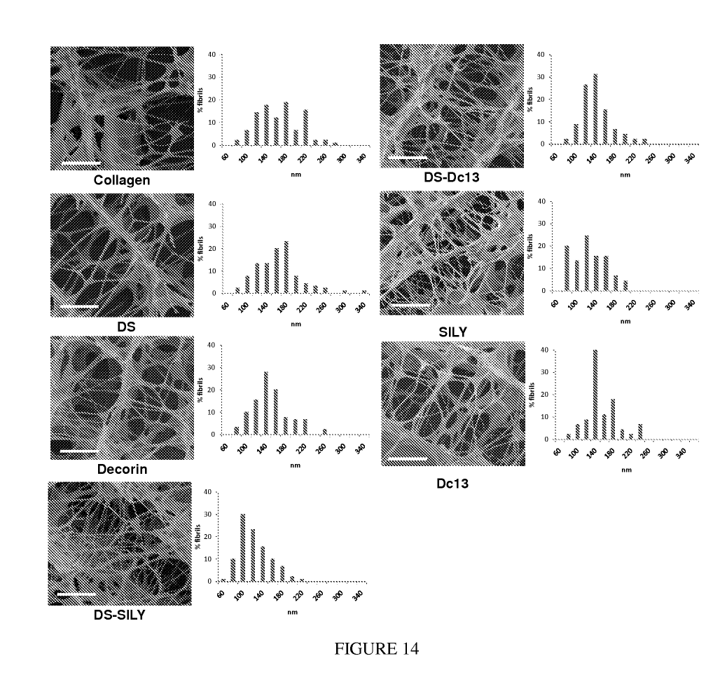

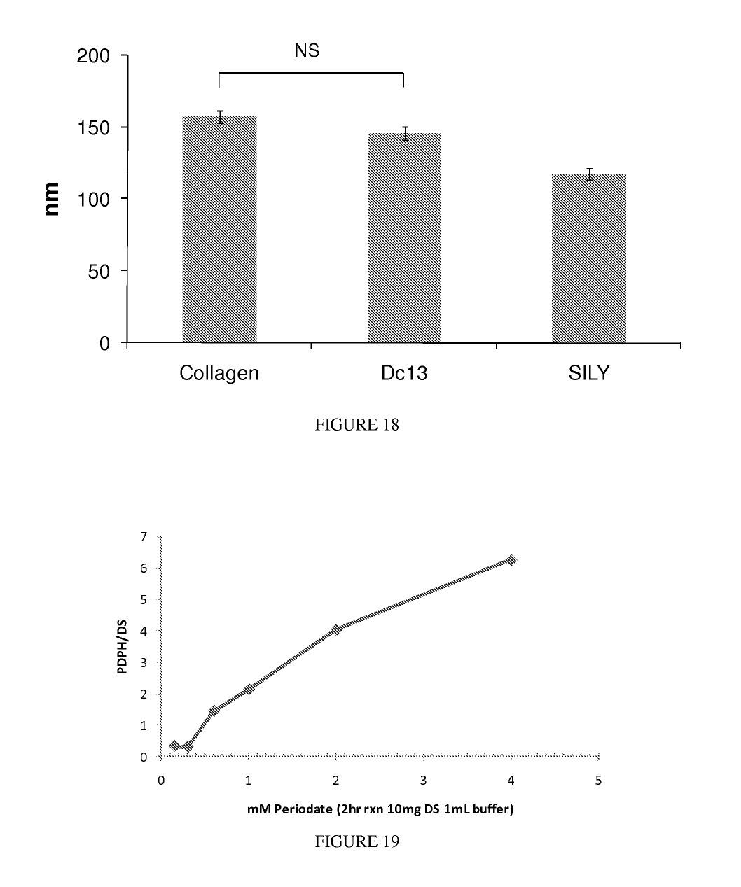

Gels for cryo-SEM were formed, as in EXAMPLE 16, directly on the SEM stage and incubated at 37° C. overnight. The stages were then secured in a cryo-holder and plunged into liquid nitrogen slush. Samples were then transferred to a Gatan Alto 2500 pre-chamber cooled to −170° C. under vacuum. A free-break surface was created with a cooled scalpel, and each sample evaporated under sublimation conditions for 20 min. The sample was coated by platinum sputter coating for 120 s. Samples were transferred to the cryo-stage at −130° C. and regions with similar orientation were imaged for comparison across treatments. Representative samples imaged at 20,000× are shown in FIG. 14. Analysis of the images was performed to determine the fibril diameter distribution, presented in histograms adjacent the corresponding image in FIG. 14, and average fibril diameter, FIGS. 17 and 18. Fibril diameter was calculated using ImageJ software (NIH) measuring individual fibrils by hand (drawing a line across fibrils and measuring its length after properly setting the scale). At least 45 independent fibril measurement were recorded for average fibril diameter calculations. A significant decrease in average fibril diameter was observed with the addition of decorin, peptidoglycans DS-SILY and DS-Dc13, and free SILY peptide.

### Example 22

### Cryo-SEM Measurements on Collagen III

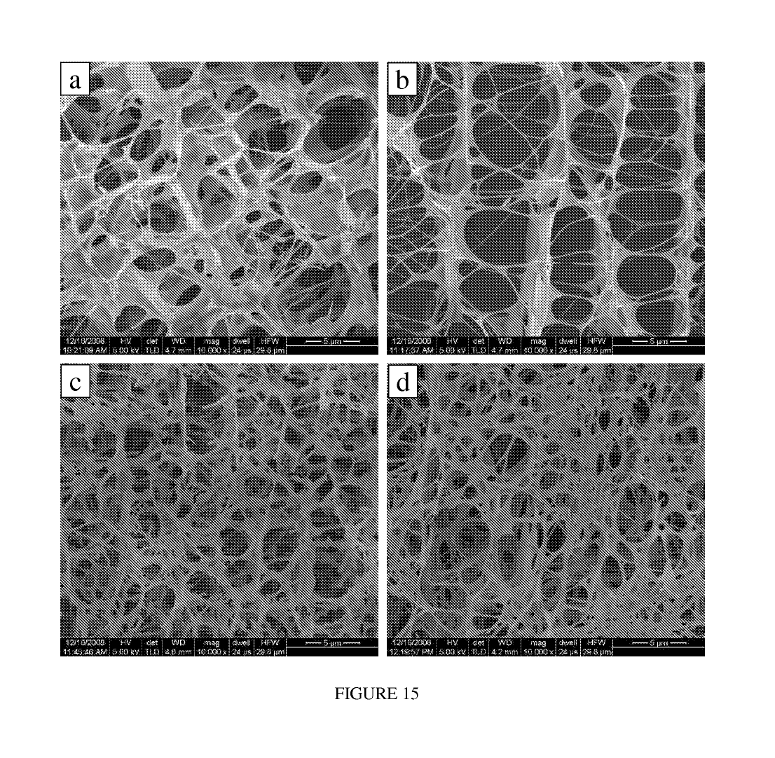

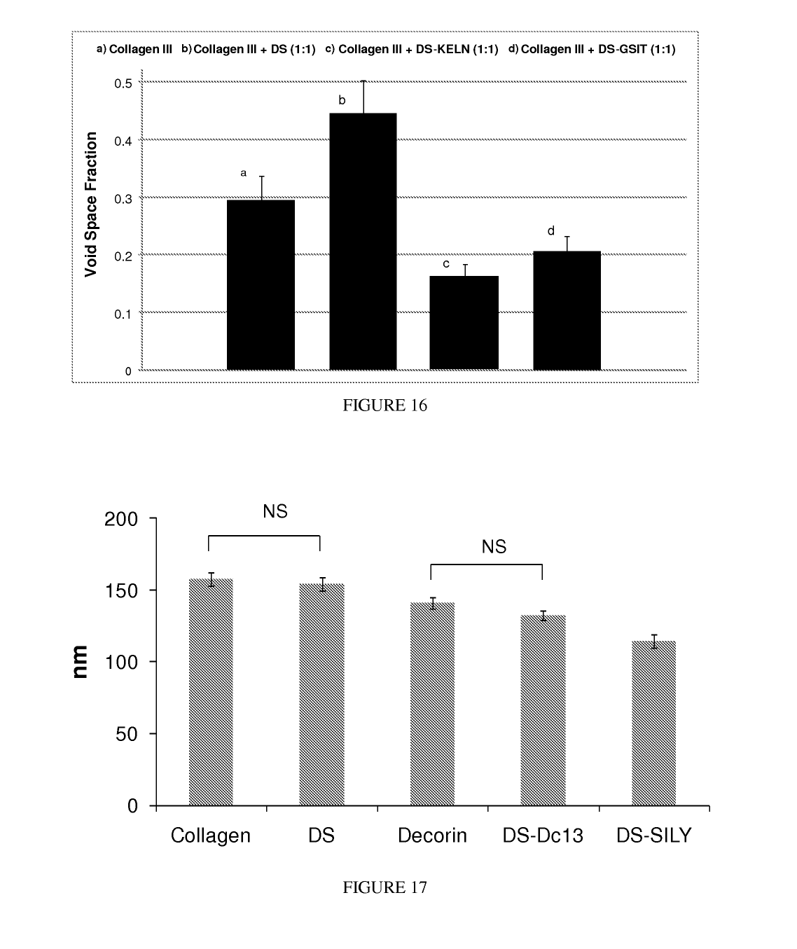

Gels for cryo-SEM were formed, as in EXAMPLE 16, directly on the SEM stage and incubated at 37° C. overnight with the following modifications. The collagen concentration was 1 mg/mL (90% collagen III, 10% collagen I). The collagen:DS ratio was 1:1 and the collagen:peptidoglycan ratio was 1:1. The images were recorded as in EXAMPLE 21. The ratio of void volume to fibril volume was measured using a variation of the method in EXAMPLE 21. The results are shown in FIGS. 15 and 16. Dermatan sulfate-KELN and dermatan sulfate-GSIT decrease void space (increase fibril diameter and branching) in the treated collagen gels.

### Example 23

### AFM Confirmation of D-Banding

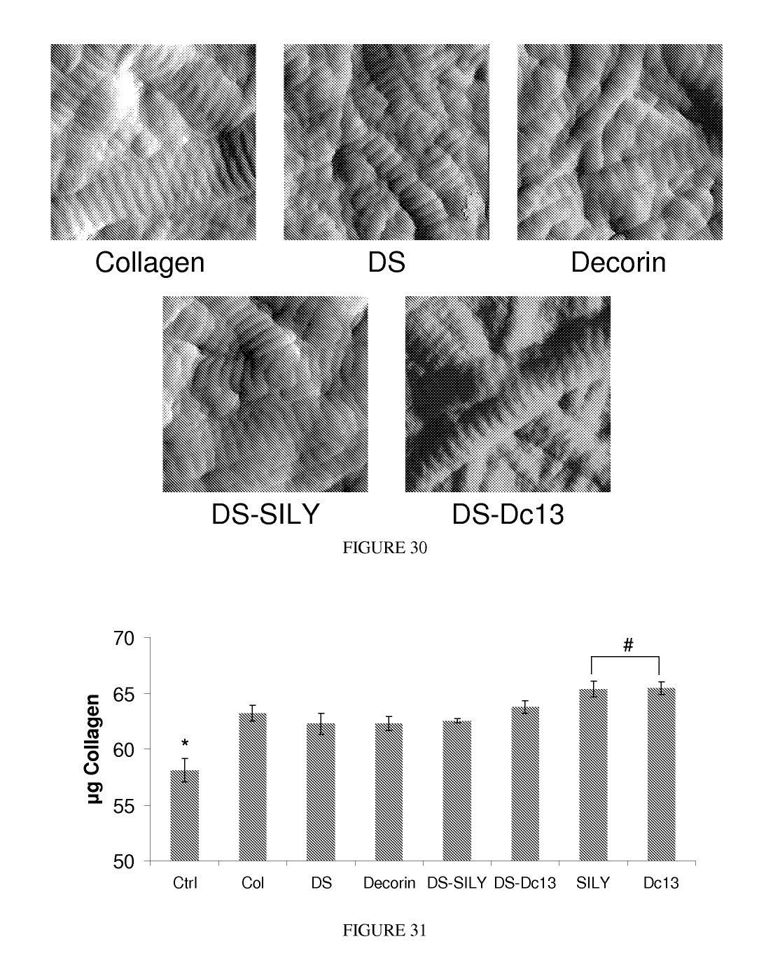

Gel solutions were prepared as described in EXAMPLE 16 and 20 μL of each sample were pipetted onto a glass coverslip and allowed to gel overnight in a humidified incubator. Gels were dehydrated by treatment with graded ethanol solutions (35%, 70%, 85%, 95%, 100%), 10 min in each solution. AFM images were made in contact mode, with a scan rate of 2 Hz (Multimode SPM, Veeco Instruments, Santa Barbara, Calif., USA, AFM tips Silicon Nitride contact mode tip k=0.05N/m, Veeco Instruments) Deflection setpoint: 0-1 Volts (FIG. 30). D-banding was confirmed in all treatments as shown in FIG. 31.

### Example 24

### Collagen Remodeling

Tissue Sample Preparation

Following a method by Grassl, et al. (Grassl, et al., Journal of Biomedical Materials Research 2002, 60, (4), 607-612), which is herein incorporated in its entirety, collagen gels with or without synthetic PG mimics were formed as described in EXAMPLE 16. Human aortic smooth muscle cells (Cascade Biologics, Portland, Oreg.) were seeded within collagen gels by adding 4×10⁶ cells/mL to the neutralized collagen solution prior to incubation. The cell-collagen solutions were pipetted into an 8-well Lab-Tek chamber slide and incubated in a humidified 37° C. and 5% CO₂ incubator. After gelation, the cell-collagen gels will be covered with 1 mL Medium 231 as prescribed by Cascade. Every 3-4 days, the medium was removed from the samples and the hydroxyproline content measured by a standard hydroxyproline assay (Reddy, 1996).

Hydroxyproline Content

To measure degraded collagen in the supernatant medium, the sample was lyophilized, the sample hydrolyzed in 2M NaOH at 120° C. for 20 min. After cooling, free hydroxyproline was oxidized by adding chloramine-T (Sigma) and reacting for 25 min at room temperature. Ehrlich's aldehyde reagent (Sigma) was added and allowed to react for 20 min at 65° C. and followed by reading the absorbance at 550 nm on an M-5 spectrophotometer (Molecular Devices). Hydroxyproline content in the medium is an indirect measure degraded collagen and tissue remodeling potential. Cultures were incubated for up to 30 days and three samples of each treatment measured. A gels incubated without added cells were used as a control. Free peptides SILY and Dc13 resulted in greater collagen degradation compared to collagen alone as measured by hydroxyproline content in cell medium as shown in FIG. 31.

Cell Viability

Cell viability was determined using a live/dead violet viability/vitality kit (Molecular Probes. The kit contains calcein-violet stain (live cells) and aqua-fluorescent reactive dye (dead cells). Samples were washed with 1×PBS and incubated with 3004, of dye solution for 1 hr at room temperature. To remove unbound dye, samples were rinsed with 1×PBS. Live and dead cells were counted after imaging a 2-D slice with filters 400/452 and 367/526 on an Olympus FV1000 confocal microscope with a 20× objective. Gels were scanned for representative regions and 3 image sets were taken at equal distances into the gel for all samples.

### Example 25

### Preparation of DS-Dc13

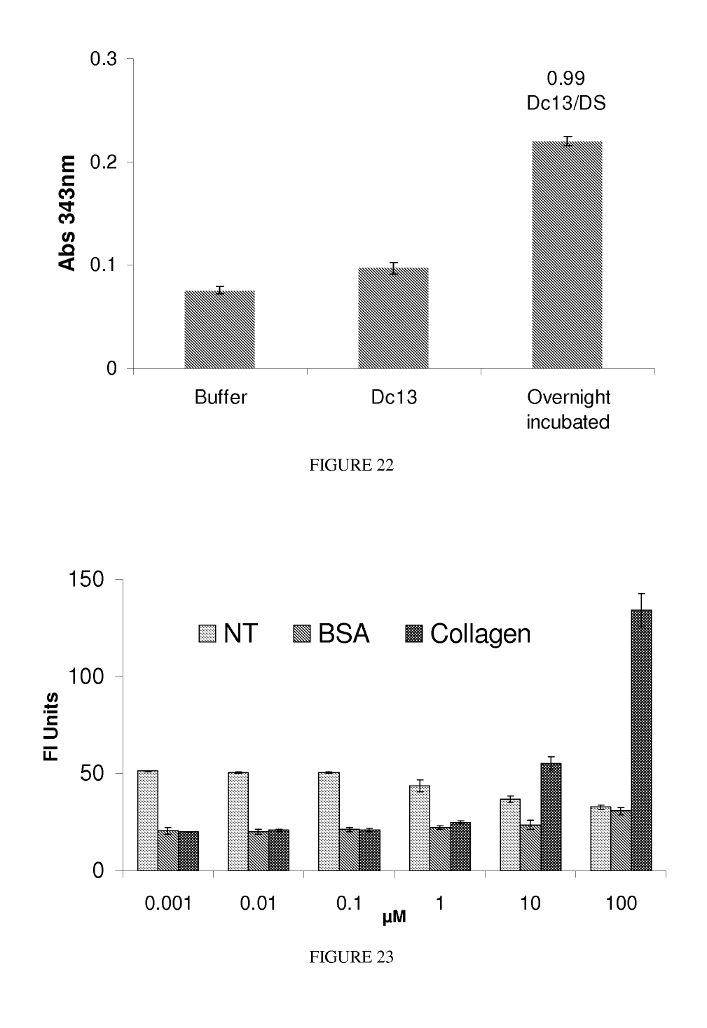

The Dc13 peptide sequence is SYIRIADTNITGC and its fluorescently labeled form is ZSYIRIADTNITGC, where Z designates dansylglycine. Conjugation to dermatan sulfate using the heterobifunctional crosslinker PDPH is performed as described for DS-SILY in EXAMPLE 3. As shown in FIG. 22, the molar ratio of Dc13 to dermatan sulfate in the conjugate (DS-Dc13) was about 1.

### Example 26

### Fluorescence Binding Assay For DS-ZSILY

The fluorescence binding assays described for DS-ZSILY was performed with peptide sequence ZSYIRIADTNITGC (ZDc13). The results appear in FIG. 23, showing that DS-ZDc13 binds specifically to the collagen surface in a dose-dependent manner, though saturation was not achieved at the highest rate tested.

### Example 27

### Fibrillogenesis Assay for DS-Dc13

A fibrillogenesis assay as described for DS-SILY, EXAMPLE 19, performed with the conjugate DS-Dc13. The results shown in FIG. 12 indicate that the DS-Dc13 delays fibrillogenesis and decreases overall absorbance in a dose-dependent manner. Free Dc13 peptide in contrast has little effect on fibrillogenesis compared to collagen alone at the high 1:1 collagen:additive molar ratio.

### Example 28

### Measurement of TGF-β1 Production by Human Dermal Fibroblasts

Human dermal fibroblasts (Cascade Biologics) were seeded onto 96-well tissue culture polystyrene plates at a seeding density of 1.83×10³ cells/well. Cells adhered overnight and cell medium was aspirated. 100 μL/well of cell medium containing a final concentration of 1.4 μM treatment delivered from a concentrated solution of treatment in 1×PBS was added to the cells. After 48 hours, cell medium was removed and frozen at −80 C until further testing. TGF-β1 was measured by ELISA using a kit and protocol from R&D Systems. Cells treated with decorin, peptidoglycan DS-SILY, dermatan sulfate, and SILY peptide significantly decreased TGF-β1 as shown in FIG. 37.

### Example 29

### Cell Culture and Gel Compaction

Human coronary artery smooth muscle cells (HCA SMC) (Cascade Biologics) were cultured in growth medium (Medium 231 supplemented with smooth muscle growth factor). Cells from passage 3 were used for all experiments. Differentiation medium (Medium 231 supplemented with 1% FBS and 1× pen/strep) was used for all experiments unless otherwise noted. This medium differs from manufacturer protocol in that it does not contain heparin.

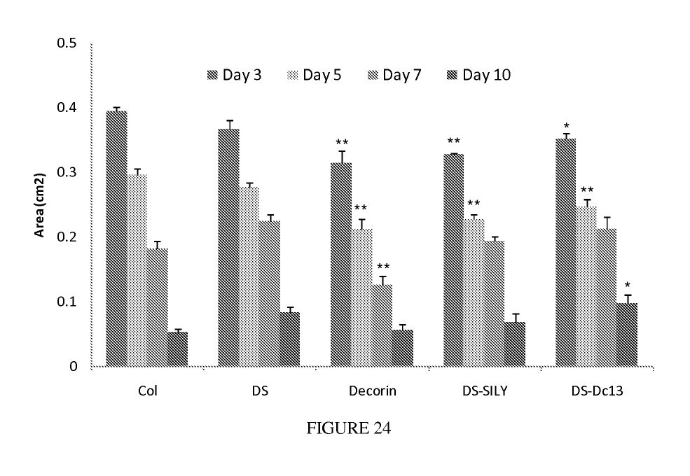

Collagen gels were prepared with each additive as described with the exception that the 1×PBS example addition was omitted to accommodate the addition of cells in media. After incubating on ice for 30 min, HCA SMCs in differentiation medium were added to the gel solutions to a final concentration of 1×10⁶ cells/mL. Gels were formed in quadruplicate in 48-well non-tissue culture treated plates (Costar) for 6 hrs before adding 500 μL/well differentiation medium. Gels were freed from the well edges after 24 hrs. Medium was changed every 2-3 days and images for compaction were taken at the same time points using a Gel Doc System (Bio-Rad). The cross-sectional area of circular gels correlating to degree of compaction was determined using ImageJ software (NIH). Gels containing no cells were used as a negative control and cells in collagen gels absent additive were used as a positive control. The results are shown in FIG. 24. At early time points, decorin and peptidoglycans DS-SILY and DS-Dc13 significantly compacted more than gels made of collagen alone or collagen with dermatan sulfate. By day 10 all gels had compacted to approximately 10% of the original gel area, and differences between additives were small. Gels treated with DS-Dc13 were slightly, but significantly, less compact than gels treated with decorin or collagen but compaction was statistically equivalent to that seen with DS and DS-SILY treated gels.

### Example 30

### Measurement of Elastin

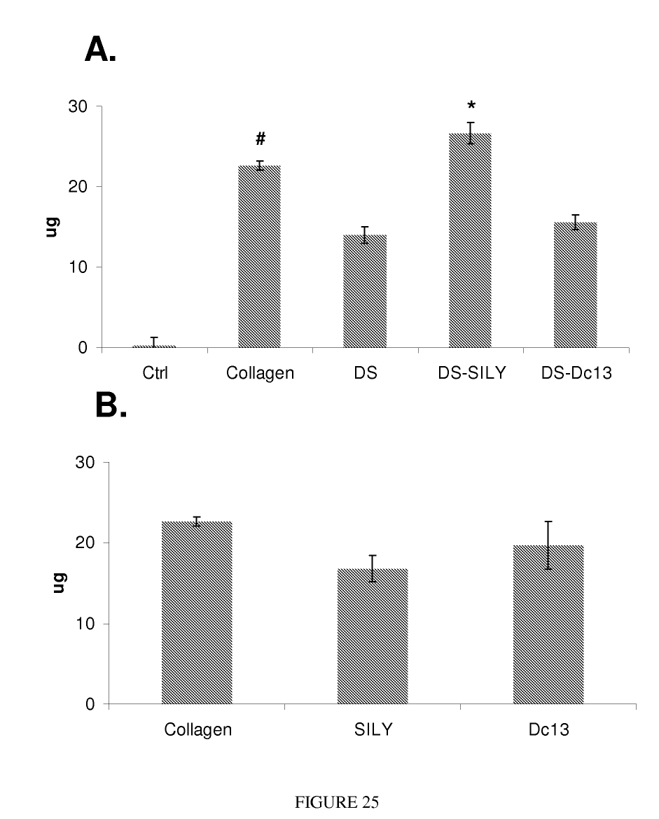

Collagen gels seeded with HCA SMCs were prepared as described in EXAMPLE 30. Differentiation medium was changed every three days and gels were cultured for 10 days. Collagen gels containing no cells were used as a control. Gels were rinsed in 1×PBS overnight to remove serum protein, and gels were tested for elastin content using the Fastin elastin assay per manufacturers protocol (Biocolor, County Atrim, U.K.). Briefly, gels were solubilized in 0.25 M oxalic acid by incubating at 100° C. for 1 hr. Elastin was precipitated and samples were then centrifuged at 11,000×g for 10 min. The solubilized collagen supernatant was removed and the elastin pellet was stained by Fastin Dye Reagent for 90 min at room temperature. Samples were centrifuged at 11,000×g for 10 min and unbound dye in the supernatant was removed. Dye from the elastin pellets was released by the Fastin Dye Dissociation Reagent, and 100 μL samples were transferred to a 96-well plate (Costar). Absorbance was measured at 513 nm, and elastin content was calculated from an α-elastin standard curve. The results of these assays are shown in FIG. 25. Treatment with DS-SILY significantly increased elastin production over all samples. Treatment with DS and DS-Dc13 significantly decreased elastin production over untreated collagen. Control samples of collagen gels with no cells showed no elastin production.

### Example 31

### Cryo-SEM Measurement of Fibril Density

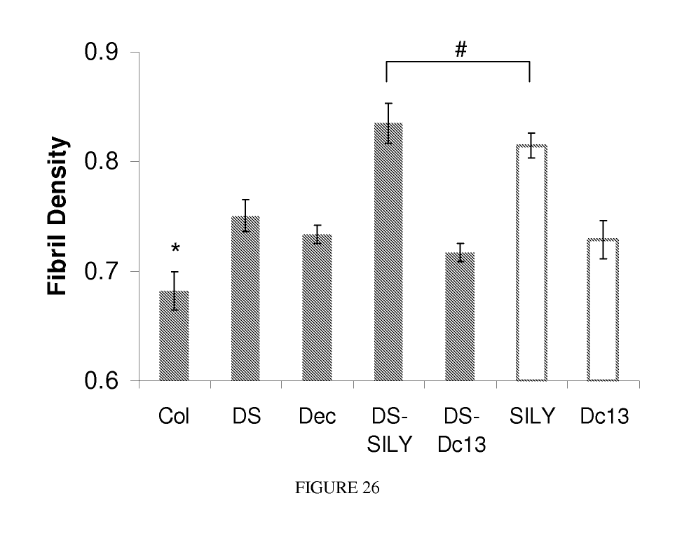

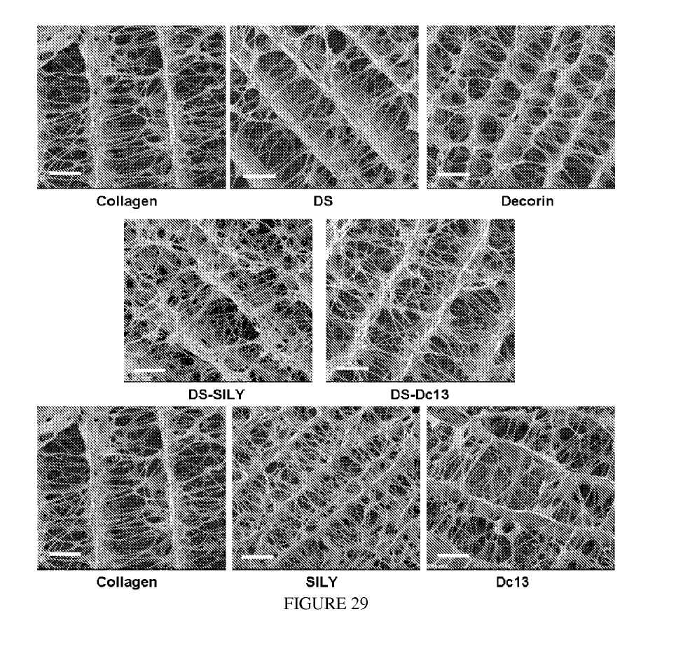

Collagen gels were formed in the presence of each additive at a 10:1 molar ratio, as described in EXAMPLE 16, directly on the SEM stage, processed, and imaged as described. Images at 10,000× were analyzed for fibril density calculations. Images were converted to 8-bit black and white, and threshold values for each image were determined using ImageJ software (NIH). The threshold was defined as the value where all visible fibrils are white, and all void space is black. The ratio of white to black area was calculated using MatLab software. All measurements were taken in triplicate and thresholds were determined by an observer blinded to the treatment. Images of the gels are shown in FIG. 29 and the measured densities are shown in FIG. 26.

### Example 32

### Viscoelastic Characterization of Gels Containing Dc13 or DS-Dc13

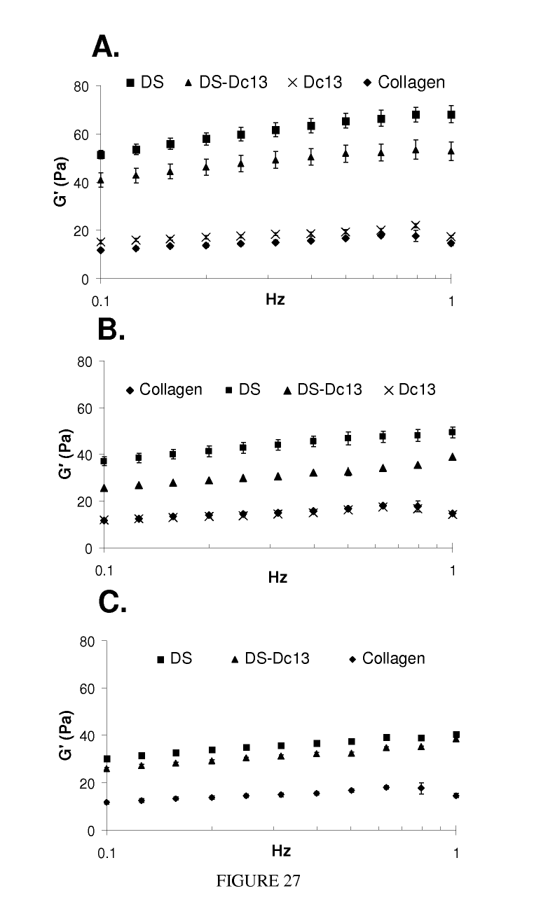

Collagen gels were prepared, as in EXAMPLE 16. Viscoelastic characterization was performed as described in EXAMPLE 17 on gels formed with varying ratios of collagen to additive (treatment). Treatment with dermatan sulfate or dermatan-Dc13 conjugate increase the stiffness of the resulting collagen gel over untreated collagen as shown in FIG. 27.

### Example 33

### Cell Proliferation and Cytotoxicity Assay

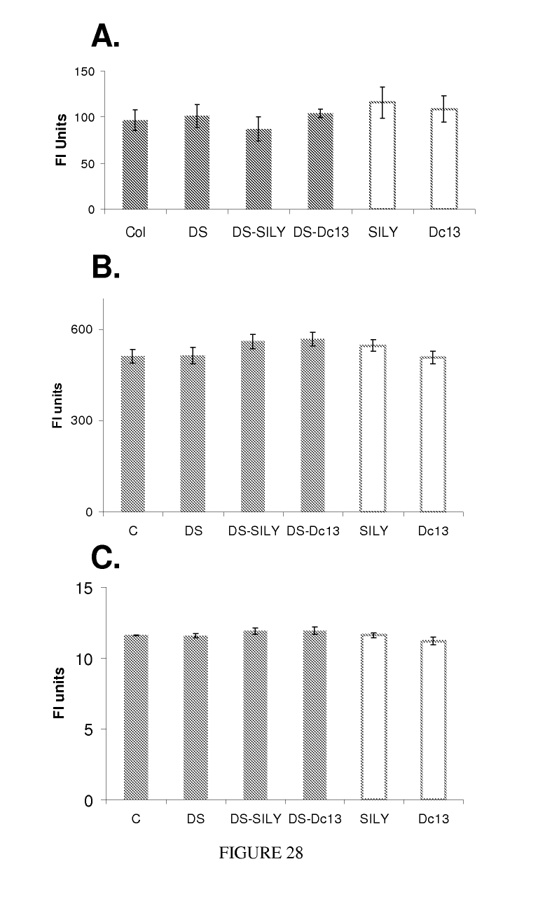

HCA SMCs, prepared as in EXAMPLE 29, were seeded at 4.8×10⁴ cells/mL in growth medium onto a 96-well tissue-culture black/clear bottom plate (Costar) and allowed to adhere for 4 hrs. Growth medium was aspirated and 600 μL of differentiation medium containing each additive at a concentration equivalent to the concentration within collagen gels (1.4×10⁻⁶ M) was added to each well. Cells were incubated for 48 hrs and were then tested for cytotoxicity and proliferation using Live-Dead and CyQuant (Invitrogen) assays, respectively, according to the manufacturer's protocol. Cells in differentiation medium containing no additive were used as control. The results are shown in FIG. 28 indicating that none of the treatments demonstrated significant cytotoxic effects.

### Example 34

### Materials

The collagen-binding peptidoglycan DS-SILY was synthesized as described in which a single SILY peptide was conjugated to DS (Paderi, J. E., and Panitch, A. Design of a Synthetic Collagen-Binding Peptidoglycan that Modulates Collagen Fibrillogenesis. Biomacromolecules 9, 2562, 2008; incorporated herein by reference). Sodium hyaluronate (Hyacoat, MW>1×10⁶ DA, 10 mg/mL in) was purchased from Hymed (Bethlehem, Pa.). Male, Long-Evans rats, 200-25 g were purchased from Harlan Labs and were handled according to approved animal care procedures at Purdue University (PACUC). All other reagents were purchased from Sigma or VWR.

### Example 35

### Incisional Model

Using sterile techniques, longitudinal wounds were incised on shaved dorsal skin of the rats. A 4 cm incision was cut through the panniculus down to the skeletal musculature, and a 250 μL single dose of either 10 mg/mL hyaluronic acid (HA) or HA+DS-SILY was applied to the open wound by a syringe. DS-SILY was tested at 0.5, 1, and 2.5 mg/mL mixed with HA. The incision was then sutured closed and animals were returned to individual cages and monitored for complications. Negative control rats received no treatment and were treated identically. A pilot study was performed (n=3) at time points 3, 7, 10, 14, and 21 days, followed by a higher powered study (n=9) for 21 and 28 day time points.

At a predetermined time after surgery (3-28 days) the animals were euthanized. Photographs of the incision were obtained, and the dermal wound including a 1 inch area around the wound edge was excised and cut into 4 mm wide strips using a custom cutting device with fixed blades. Relevant tissues were harvested for tensile strength testing and histologic study.

The pilot study (n=3 rats/treatment) demonstrated that the addition of DS-SILY at both a low (0.125 mg) and high (0.625 mg) dose significantly increases scar strength at the later time point 21-days. Based on these findings, a full powered (n=9 rats/treatment) was performed using the later time points 21-day and 28-day, and comparing the same low dose, but modifying the high dose to 0.25 mg DS-SILY. DS-SILY was delivered with HA in each study and the negative control received no treatment.

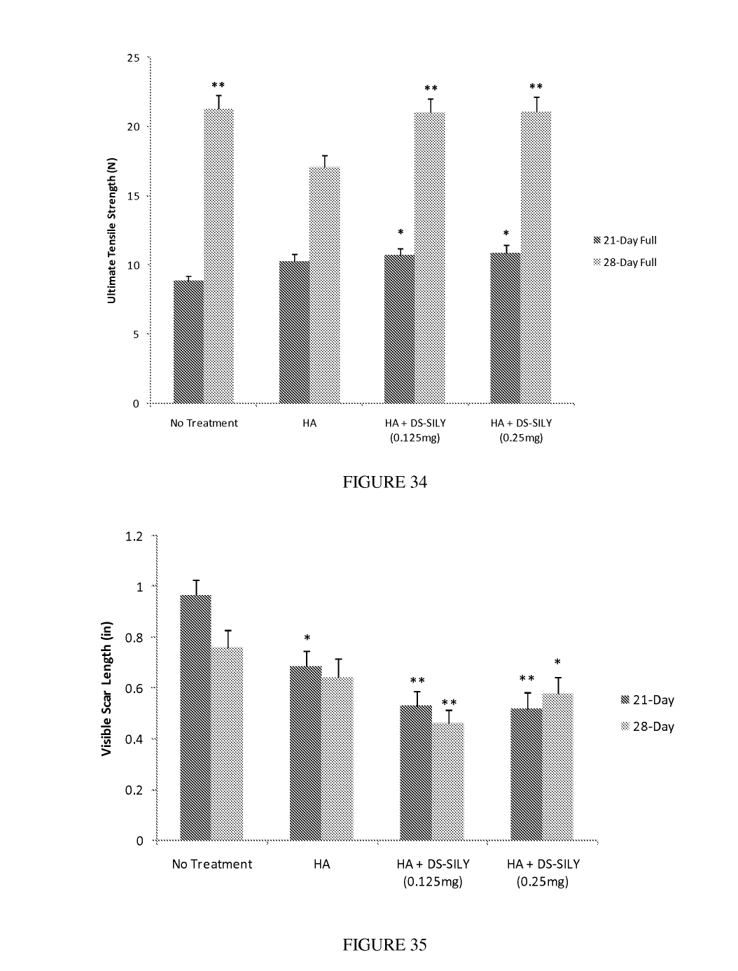

DS-SILY increased scar strength over no treatment at 21-days, indicating a more rapid healing time. At 28-days, the scar strength was significantly higher compared to the HA control, but was not different from no treatment. At this time point, HA has a negative effect on wound strength, as it results in a significantly weaker scar compared to no treatment. The addition of DS-SILY at either dose however, overcomes the negative effects of HA as seen by the increase in scar strength. Results are shown in FIG. 34.

### Example 36

### Tensile Testing

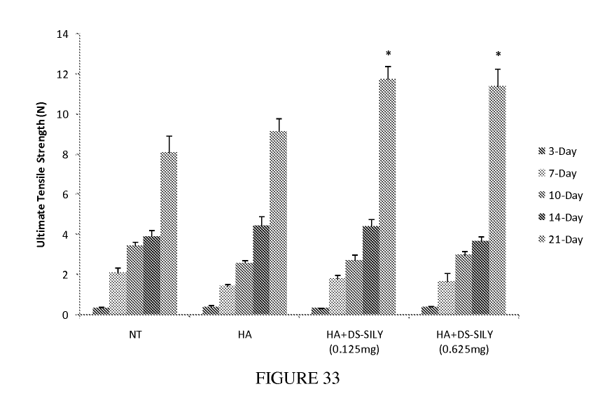

Following necropsy, 4 mm skin strips (n=4 per animal) were placed in 1×PBS and kept at 4° C. for up to 6 hours. The wound breaking strength was measured at time points from 3 to 21 days. Skin samples were loaded onto a mechanical testing system (Test Resources, model: 100P/Q) such that the incision was orthogonal to the grips. Samples were loaded under tension with a rate of 5 mm/min to failure. Results are shown in FIG. 33. As shown in FIG. 33, at 21 days post-injury, peptidoglycan treated wounds were significantly stronger, with a significant increase in wound breaking strength when compared to untreated or HA treated wounds. HA treatment showed a modest, but not significant increase in wound strength over untreated wounds. No differences were observed between the low and high peptidoglycan concentrations.

### Example 37

### Histological Study

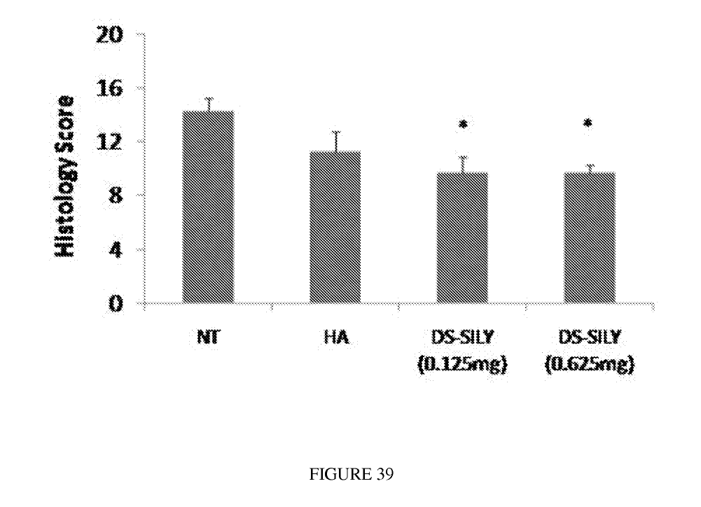

Skin strips 4 mm wide were fixed in 10% formalin solution following necropsy, and were embedded and sectioned for H&E and Masson's trichrome staining (FIG. 39). Immunological markers were graded following the methods of Simhon et. al (Simhon, D., Ravid, A., Halpern, M., Cilesiz, I., Brosh, T., Kariv, N., Leviav, A., and Katzir, A. Laser soldering of rat skin, using fiberoptic temperature controlled system. Lasers in Surgery and Medicine 29, 265, 2001; incorporated herein by reference), and ECM organization was graded following the methods of Beausang et. al. (Beausang, E., Floyd, H., Dunn, K. W., Orton, C. I., and Ferguson, M. W. J. A new quantitative scale for clinical scar assessment. Meeting of the European-Tissue-Repair-Society. Cologne, Germany, 1997, pp. 1954-1961; incorporated herein by reference).

H&E stained samples were examined for inflammation by a board certified pathologies blinded to the treatments following a scale adapted from Simhon et al. Trichrome stained samples were evaluated for scar tissue formation by an observer blinded to the treatments using a method established by Beausang et al. in which collagen orientation, density, and maturation are observed and compared to collagen of healthy tissue. A total of 12 tissue samples were analyzed for each study at each time point. Statistics were analyzed by ANOVA using Design Expert software (StatEase, Minneapolis, Minn.). Results are presented as average+S.E. and significance was set by α=0.05.

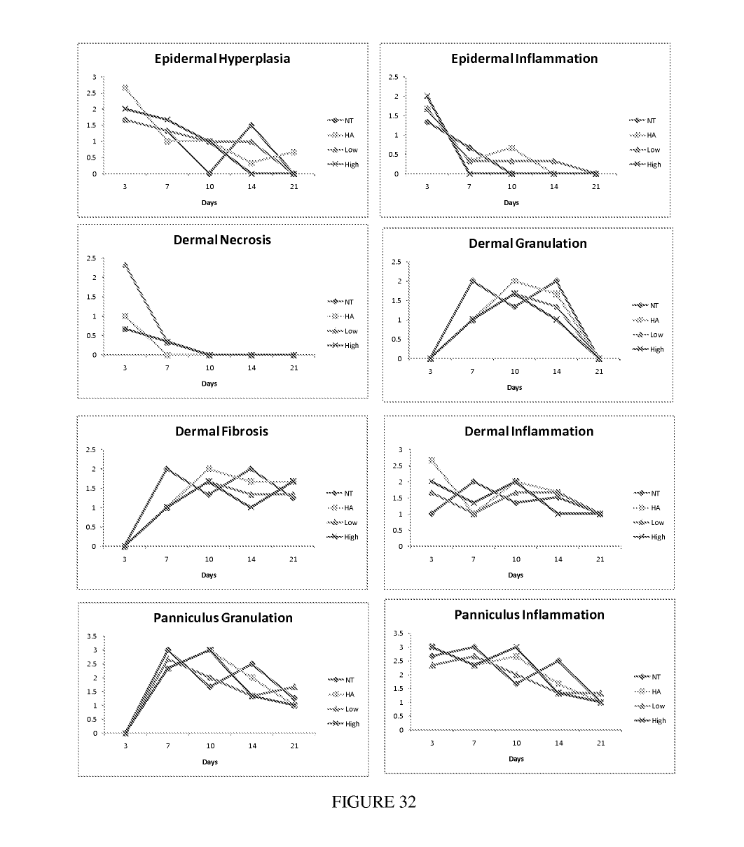

As shown in FIG. 32, treatment with both low and high peptidoglycan doses did not have any adverse inflammatory effect, and no significant differences were found with between any treatment groups. By 21-days, inflammation had subsided and remodeling of the newly synthesized tissue had begun.

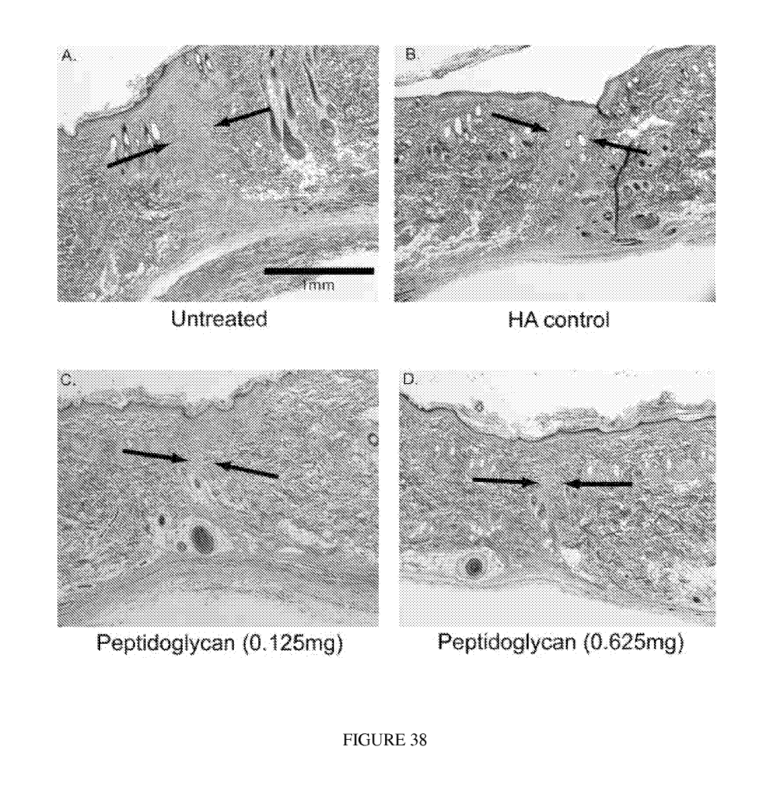

Improved tissue maturity and organization, and scar-free healing were seen with peptidoglycan treatments. Wounds at 21-days post-injury were trichrome stained and assessed for scar tissue formation using methods which evaluate collagen organization, maturity and density (Beausang). FIG. 38 shows representative histological sections of tissues with different treatment types. In untreated and HA treated wounds, typical scar tissue marked by dense and immature collagen was seen in the wounded areas. In contrast, peptidoglycan treated wounds showed significantly less scar tissue. This visual observation is supported by histological scoring, presented in FIG. 39. Both peptidoglycan treatments received significantly lower scores, indicating more normal or scar-free tissue compared to untreated wounds. HA treated wounds show a modest decrease in histological score, which is not significant compared to untreated wounds.

### Example 38

### Visual Scar Scoring

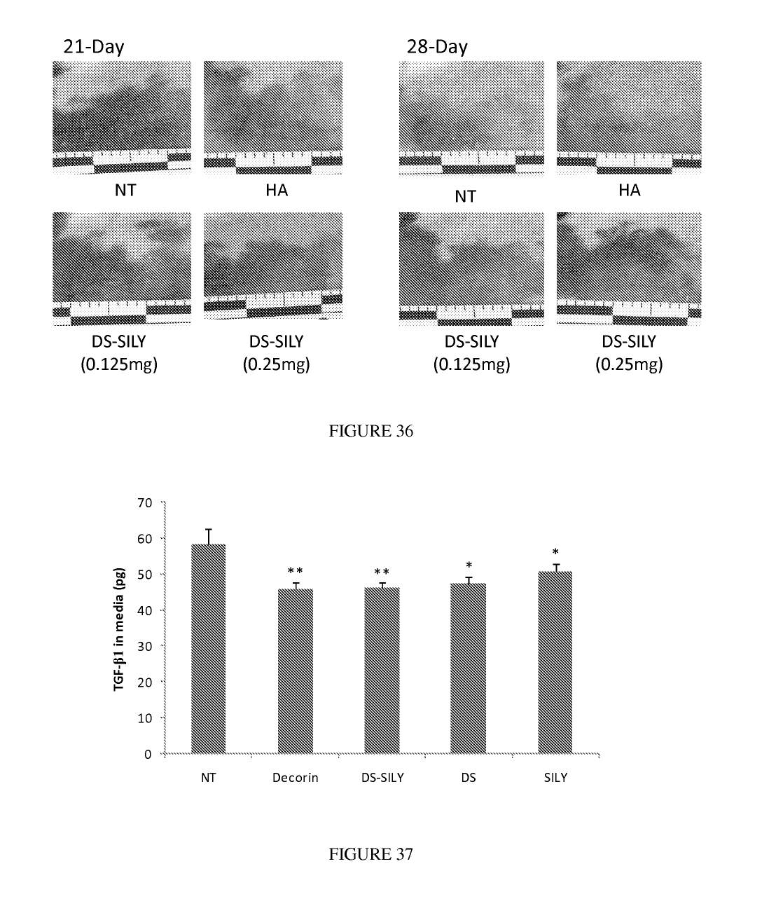

Photographs of scars were taken at the time of necropsy using a digital camera with predetermined manual settings mounted on a camera stand to standardize focal distance (FIG. 36). A scale bar was included in each image and was used to determine the visible scar length. At the 21 and 28-day times points, five blinded observers traced the visible scar length using ImageJ software (NIH) to give a quantitative measure of visual scar healing. Results are shown in FIG. 35.

### Example 39

### Peptidoglycan Synthesis

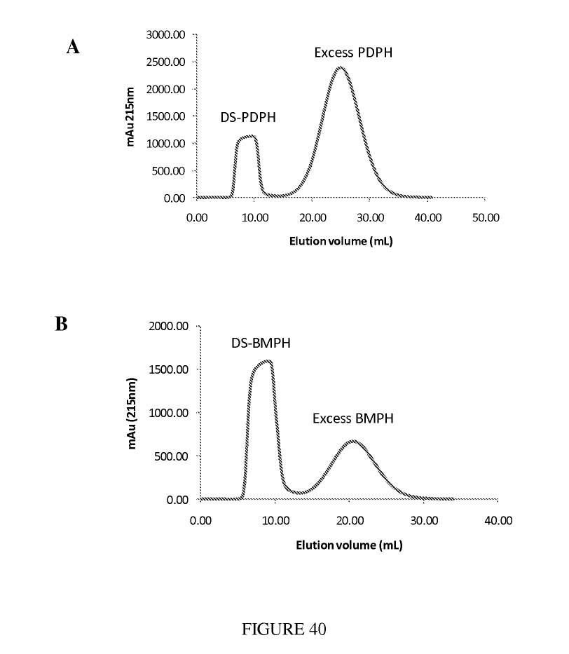

The peptidoglycan was synthesized as described with modifications. Dermatan sulfate (DS) was oxidized by periodate oxidation in which the degree of oxidation was controlled by varying amounts of sodium meta-periodate. After oxidizing at room temperature for 2 hours protected from light, the oxidized DS was desalted into 1×PBS pH 7.2 by size exclusion chromatography using a column packed with Bio-gel P-6 (BioRad). The heterobifunctional crosslinkers either PDPH or BMPH was added to oxidized DS in 30 fold molar excess to DS, and was reacted for 2 hours at room temperature protected from light. The intermediate product DS-crosslinker was then purified of excess crosslinker by size exclusion as described with 1×PBS pH 7.2 as running buffer and shown in FIG. 40, Panels A and B, for PDPH and BMPH, respectively. The number of crosslinkers attached to DS was calculated by the consumption of crosslinker determined from the 215 nm peak area of the excess crosslinker peak. A standard curve of crosslinker was generated to calculate excess crosslinker. The free peptide SILY was dissolved into water at a concentration of 2 mg/mL and was added in 1 molar excess to the number of attached crosslinkers and was reacted for 2 hours at room temperature. The final product DS-SILYₙ was purified by size exclusion using a column packed with Sephadex G-25 medium (GE Lifesciences) with Millipore water as the running buffer. The final product was immediately frozen, lyophilized, and stored at −20 C until further testing.

### Example 40

### Peptidoglycan Preparation and Delivery for Wound Healing

The peptidoglycan DS-PDPH-SILY₄ was prepared as described. After lyophilization, the peptidoglycan was weighed and dissolved to a final concentration of 1 mg/mL into Millipore water containing 30 mg/mL D-mannitol (Sigma). The solution was then sterile filtered using a 0.22 um syringe filter. Under sterile conditions, 250 μL of filtered solution were aliquotted into 1.5 mL lobind tubes and were frozen and lyophilized. For use in the previously described incisional rat model, 250 μL of HA (Hycoat) was mixed with the lyophilized peptidoglycan/mannitol and was applied to the open wounds.

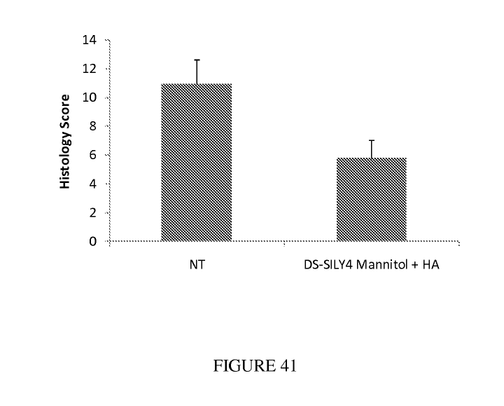

After 28 days post wounding, rats were sacrificed and the wound tissue was harvested for evaluation. Histological evaluation following a previously described scoring system was performed. As shown in FIG. 41, the peptidoglycan treated wounds resulted in a significant improvement (p<0.05) over untreated wounds.

1. A method of promoting wound healing in a patient, said method comprising the steps of
administering to the patient a collagen-binding synthetic peptidoglycan, wherein the collagen-binding synthetic peptidoglycan promotes healing of a wound in the patient.

2. (canceled)

3. The method of claim 1 wherein the collagen-binding synthetic peptidoglycan is in the form of an engineered collagen matrix wherein the collagen-binding synthetic peptidoglycan is incorporated into the engineered collagen matrix.

4-5. (canceled)

6. The method of claim 3 wherein the molar ratio of the collagen to the collagen-binding synthetic peptidoglycan is from about 1:1 to about 40:1.

7-8. (canceled)

9. The method claim 1 wherein the collagen-binding synthetic peptidoglycan has amino acid homology with a portion of the amino acid sequence of a proteoglycan or a protein that regulates collagen fibrillogenesis.

10. The method of claim 1 wherein the collagen-binding synthetic peptidoglycan has amino acid homology with a portion of a collagen-binding protein that does not regulate collagen fibrillogenesis.

11. The method of claim 3 wherein the matrix further comprises an exogenous population of cells.

12. (canceled)

13. The method of claim 3 wherein the matrix further comprises at least one polysaccharide.

14. The method of claim 1 wherein the collagen-binding synthetic peptidoglycan is a compound of formula PₙGₓ wherein n is 1 to 30;
wherein x is 1 to 10;
P is a synthetic peptide of about 5 to about 40 amino acids comprising a sequence of a collagen-binding domain; and
G is a glycan.

15. The method of claim 1 wherein the collagen-binding synthetic peptidoglycan is a compound of formula (PₙL)ₓG wherein n is 1 to 5;
wherein x is 1 to 10;
P is a synthetic peptide of about 5 to about 40 amino acids comprising a sequence of a collagen-binding domain;
L is a linker; and
G is a glycan.

16. The method of claim 1 wherein the collagen-binding synthetic peptidoglycan is a compound of formula P(LGₙ)ₓ wherein n is 1 to 5;
x is 1 to 10;
P is a synthetic peptide of about 5 to about 40 amino acids comprising a sequence of a collagen-binding domain;
L is a linker; and
G is a glycan.

17. The method of claim 1 wherein the glycan is a glycosaminoglycan or a polysaccharide.

18-20. (canceled)

21. The method of claim 1 wherein the glycan is dermatan sulfate.

22. The method of claim 1 wherein the peptide comprises the amino acid sequence RRANAALKAGELYKSILYGC.

23-27. (canceled)

28. A method of decreasing scar formation in a patient, said method comprising the steps of
administering to the patient a collagen-binding synthetic peptidoglycan, wherein the collagen-binding synthetic peptidoglycan decreases scar formation in the patient.

29. (canceled)

30. The method of claim 28 wherein the collagen-binding synthetic peptidoglycan is in the form of an engineered collagen matrix wherein the collagen-binding synthetic peptidoglycan is incorporated into the engineered collagen matrix.

31-32. (canceled)

33. The method of claim 30 wherein the molar ratio of the collagen to the collagen-binding synthetic peptidoglycan is from about 1:1 to about 40:1.

34-35. (canceled)

36. The method of claim 28 wherein the collagen-binding synthetic peptidoglycan has amino acid homology with a portion of the amino acid sequence of a proteoglycan or a protein that regulates collagen fibrillogenesis.

37. The method of claim 28 wherein the collagen-binding synthetic peptidoglycan has amino acid homology with a portion of a collagen-binding protein that does not regulate collagen fibrillogenesis.

38. The method of claim 30 wherein the matrix further comprises an exogenous population of cells.

39. (canceled)

40. The method of claim 30 wherein the matrix further comprises at least one polysaccharide.

41. The method of claim 28 wherein the collagen-binding synthetic peptidoglycan is a compound of formula PₙGₓ wherein n is 1 to 30;
wherein x is 1 to 10;
P is a synthetic peptide of about 5 to about 40 amino acids comprising a sequence of a collagen-binding domain; and
G is a glycan.

42. The method of claim 28 wherein the collagen-binding synthetic peptidoglycan is a compound of formula (PₙL)ₓG wherein n is 1 to 5;
wherein x is 1 to 10;
P is a synthetic peptide of about 5 to about 40 amino acids comprising a sequence of a collagen-binding domain;
L is a linker; and
G is a glycan.

43. The method of claim 28 wherein the collagen-binding synthetic peptidoglycan is a compound of formula P(LGₙ)ₓ wherein n is 1 to 5;
x is 1 to 10;
P is a synthetic peptide of about 5 to about 40 amino acids comprising a sequence of a collagen-binding domain;
L is a linker; and
G is a glycan.

44. The method of claim 28 wherein the glycan is a glycosaminoglycan or a polysaccharide.

45-47. (canceled)

48. The method of claim 28 wherein the glycan is dermatan sulfate.

49. The method of claim 28 wherein the peptide comprises the amino acid sequence RRANAALKAGELYKSILYGC.

50-54. (canceled)
